SEQUENCE LISTING (16 sequences)

SEQ ID NO: 1 - DNA, 21 bp, Homo sapiens
ggacccgcuuauuuauagatt

SEQ ID NO: 2 - DNA, 21 bp, Homo sapiens
ucuauaaauaagcgggucctg

SEQ ID NO: 3 - DNA, 21 bp, Homo sapiens
ccauccgcugguucaaaaatt

SEQ ID NO: 4 - DNA, 21 bp, Homo sapiens
uuuuugaaccagcggauggtg

SEQ ID NO: 5 - DNA, 21 bp, Homo sapiens
cguguauucuauuuugcgutt

SEQ ID NO: 6 - DNA, 21 bp, Homo sapiens
acgcaaaauagaauacacgtg

SEQ ID NO: 7 - DNA, 21 bp, Homo sapiens
cguacuugguagacaugcatt

SEQ ID NO: 8 - DNA, 21 bp, Homo sapiens
ugcaugucuaccaaguacgtg

SEQ ID NO: 9 - DNA, 21 bp, Homo sapiens
ccaaaaagcuuucugcuagtt

SEQ ID NO: 10 - DNA, 21 bp, Homo sapiens
cuagcagaaagcuuuuuggtg

SEQ ID NO: 11 - DNA, 21 bp, Homo sapiens
ccucgugaacaucucccautt

SEQ ID NO: 12 - DNA, 21 bp, Homo sapiens
augggagauguucacgaggtc

SEQ ID NO: 13 - DNA, 21 bp, Homo sapiens
ggcuaggaaagagauuuaatt

SEQ ID NO: 14 - DNA, 21 bp, Homo sapiens
uuaaaucucuuuccuagcctt

SEQ ID NO: 15 - DNA, 21 bp, Homo sapiens
ggugcacuuuuaucuuugctt

SEQ ID NO: 16 - DNA, 21 bp, Homo sapiens
gcaaagauaaaagugcacctg

(19) United States
(12) Patent Application Publication
(10) Pub. No.: US 2021/0363247 A1
(43) Pub. Date: Nov. 25, 2021

(54) Novel Treatments of Glaucoma

(71) Applicant: The Regents of the University of California, Oakland, CA (US)

(72) Inventors: Lu Chen, Berkeley, CA (US); Meng Shi, Berkeley, CA (US)

(73) Assignee: The Regents of the University of California, Oakland, CA (US)

(21) Appl. No.: 17/387,999

(22) Filed: Jul. 28, 2021

Related U.S. Application Data
(63) Continuation of application No. PCT/US20/15745, filed on Jan. 29, 2020.
(60) Provisional application No. 62/798,455, filed on Jan. 29, 2019.

(51) Int. Cl. C07K 16/28; A61K 9/00; A61K 45/06

(52) U.S. Cl. CPC C07K 16/2803; A61K 45/06; A61K 9/0014

(57) ABSTRACT

Glaucoma or pathogenic intraocular pressure is treated by locally administering to an eye in need thereof formulations of a Wnt5a receptor inhibitor.

This invention was made with government support under Grant Number EY028995 awarded by the National Institutes of Health. The government has certain rights in the invention.

### INTRODUCTION

Glaucoma is a major health problem which affects over 3 million Americans and 60 million people worldwide. It is estimated that 111.8 million people will be affected by this disease worldwide in 2040. A major risk factor for this disease is increased intraocular pressure (IOP), which can damage the optic nerve and cause permanent blindness without treatment. Currently, there is no cure for glaucoma. Existing eye drops or oral medications are of limited efficacy with many side effects, and surgeries often fail with scar formation and fibrosis.

Aqueous humor is the clear colorless liquid that fills the anterior and posterior chambers of the eye. It is produced by the ciliary body at the posterior chamber and exits the anterior chamber angle through the conventional pathway via trabecular meshwork and Schlemm's canal, and the nonconventional pathway via uveoscleral outflow. In normal eyes, a dynamic balance exists between the production and drainage of aqueous humor, maintaining IOP in the normal range.

Schlemm's canal (SC) is a circumferential channel located at the iridocorneal angle in the ocular anterior chamber. It is part of the conventional aqueous humor outflow system, which accounts for 70-90% of the total aqueous humor that drains out of the eye in human. The endothelial cell lining of Schlemm's canal is one of the primary sites of resistance to aqueous humor drainage and is a major determinant of IOP. When canal resistance increases with age or under pathological situation, IOP is elevated leading to glaucoma with irreversible optic nerve damage and vision loss. It is therefore an important target for glaucoma therapy. Recently, we provided the first evidence that Schlemm's canal expresses Prox-1, the master control gene of lymphatic formation (Truong T N, Li H, Hong Y K, Chen L. Novel characterization and live imaging of Schlemm's canal expressing Prox-1. PLoS One. 2014; 9(5):e98245).

We previously reported that Wnt5a is expressed on Schlemm's canal, its expression is regulated in response to sheer stress change, and by inhiting Wnt5a, we could effectively lower IOP in vivo. Wnt5a operates through multiple and alternative signaling pathways, depending on cell type, microenvironment, stimulus, etc. To identify other druggable targets for glaucoma we sought to determine downstream effectors in glaucoma-relevant models and ascertain their druggability for treating glaucoma.

We report here, inter alia, FZD2 (frizzled-2), FZD5 and ROR1 (Receptor Tyrosine Kinase Like Orphan Receptor 1), are expressed on SC, their expression is regulated in response to sheer stress change, Wnt5a stimulation or intervention. For example, FZD2, FZD5 and ROR1 modulation regulates SC function, such as tube formation. In SC cells cultured under pressure (or increased sheer stress), we observed increase of Wnt5a, Fzd2, Fzd5, and RoR1, when Wnt5a is down regulated, Fzd2, Fzd5 and ROR1 are downregulated as well, and when we stimulated human SC cells with Wnt5a, Fzd5 and ROR1 increased correspondingly.

Additionally, we disclose that calcium signaling is involved in SC function, and several related molecules, such as PLCB1 (phospholipase C, beta 1), PPP3R1 (Protein Phosphatase 3 Regulatory Subunit B, Alpha), NFATC3 (Nuclear Factor of Activated T Cells 3), CAMK2D (Calcium/Calmodulin Dependent Protein Kinase II Delta), are regulated in response to sheer stress change, Wnt5a stimulation or intervention. We disclose that these Wnt receptors (i.e. FZD2, FZD5, ROR1) and related molecules of calcium signaling pathways (i.e. PLCB1, PPP3R1, NFATC3, CAMK2D) provide targets to modulate SC function and treat glaucoma.

Wnt5a was known to operate through multiple and alternative signaling pathways, depending on cell type, microenvironment, stimulus, etc., and it was previously not known which, if any of these downstream effectors were operative, in a glaucoma-relevant model, and what effectors, if any, might provide druggable targets for controlling IOP.

### SUMMARY OF THE INVENTION

The invention provides methods and compositions for locally treating glaucoma or pathogenic intraocular pressure.

In an aspect the invention provides a method of treating glaucoma or pathogenic intraocular pressure, comprising administering to a person in need thereof an inhibitor of an ocular Wnt5a effector selected from: FZD2 (frizzled-2), FZD5 (frizzled-5) and ROR1 (Receptor Tyrosine Kinase Like Orphan Receptor 1); or PLCB1 (phospholipase C, beta 1), PPP3R1 (Protein Phosphatase 3 Regulatory Subunit B, Alpha), NFATC3 (Nuclear Factor of Activated T Cells 3) and CAMK2D (Calcium/Calmodulin Dependent Protein Kinase II Delta).

In embodiments:
inhibitor inhibits effector expression through genetic manipulation, such as CRISPR gene editing or siRNA;
the inhibitor inhibits the effector directly and is selected from an antibody, a small interfering peptide, and a small molecule inhibitor;
the administering step comprises locally administering the inhibitor to an eye in need thereof;
the administering step comprises delivery by eye drop or by intracameral administration or injection, subconjuctival administration or injection or intravitreal administration or injection;
the administration is topical, and the inhibitor is administered in form of a topical ophthalmic gel, ointment, suspension or solution or contact lens;
the inhibitor is a ROR1 inhibitor, such as selected from cirmtuzumab and KAN0439834, or a FZD5 inhibitor, such as selected from anti-FZD5 antibodies IgG-2919 and IgG-2921 (Steinhardt et al., Nat. Med. 2017;23:60-68), or and FZD2 inhibitor, such as selected from dFz7-21, a selective peptide (Nile et al, Nat. Chem. Biol. 2018;14:582-590, or FZD2 antibody, or an siRNA such as disclosed herein; and/or
the method further comprising administering or coadministering locally at the eye a second, different inhibitor that is an inhibitor of an ocular Wnt5a effector.

In another aspect the invention provides an ophthalmic formulation of an inhibitor of an ocular Wnt5a effector, in unit dosage form, for treating glaucoma or pathogenic intraocular pressure, the effector selected from: FZD2 (frizzled-2), FZD5 (frizzled-5) and ROR1 (Receptor Tyrosine Kinase Like Orphan Receptor 1); or PLCB1 (phospholipase C, beta 1), PPP3R1 (Protein Phosphatase 3 Regulatory Subunit B, Alpha), NFATC3 (Nuclear Factor of Activated T Cells 3) and CAMK2D (Calcium/Calmodulin Dependent Protein Kinase II Delta).

In embodiments:
the inhibitor inhibits effector expression through genetic manipulation, such as CRISPR gene editing or siRNA;
the inhibitor inhibits the effector directly and is selected from an antibody, a small interfering peptide, and a small molecule inhibitor;
the formulation is the form of a topical ophthalmic gel, ointment, suspension or solution;
the dosage form is an inhibitor-loaded contact lens, eye drop, depot or bollus;
the formulation is packaged in an eye drop dispenser;
the formulation is loaded in a syringe configured for intracameral administration or injection, subconjuctival administration or injection or intravitreal administration or injection;
the formulation further comprising excipients and features suitable for direct, topical delivery to the eye, selected from the group consisting of opthalmically suitable clarity, pH buffer, tonicity, viscosity, stability and sterility; and/or
the inhibitor is a ROR1 inhibitor, such as selected from cirmtuzumab and KAN0439834, or a FZD5 inhibitor, such as selected from anti-FZD5 antibodies IgG-2919 and IgG-2921, or and FZD2 inhibitor, such as selected from dFz7-21, a selective peptide, or FZD2 antibody, or an siRNA such as disclosed herein.

The invention encompasses all combinations of the particular embodiments recited herein. The methods may be practiced with all disclosed compositions including specific embodiments.

### BRIEF DESCRIPTION OF THE FIGURES

FIGS. 1A-G. (A-C) Real-time quantitative PCR data showing the expression levels of FZD5, ROR1 and FZD2 in human Schlemm's canal cells are regulated by sheer stress (A), Wnt5a intervention by small interference RNAs (siRNAs) (B), or Wnt5a stimulation (C). (D and E) Summarized data showing that tube formation of human Schlemm's canal cells was inhibited by FZD5 siRNA (D) or ROR1 siRNA (E), respectively. Scrambled siRNA was used as negative control. *P<0.05. (F and G) Representative images showing that tube formation of human Schlemm's canal cells are regulated by the intervention of FZD5 (F) or ROR1 (G) via siRNAs. *P<0.05.

FIGS. 2A-C. (A-C, top panels) Real-time quantitative PCR data showing the expression levels of PLCB1, PPP3R1 and NFATC3 in human Schlemm's canal cells are regulated by sheer stress (A), Wnt5a intervention via siRNAs (B), or Wnt5a stimulation (C). (A-C, lower panels) Representative images and summarized data of Fluo-4 staining showing intracellular calcium signal is regulated by sheer stress (A), Wnt5a intervention via siRNAs (B), or Wnt5a stimulation (C). Green: Fluo-4, blue: DAPI nuclear staining *P<0.05.

FIGS. 3A-B. Real-time quantitative PCR data showing the expression of CAMK2D in human Schlemm's canal cells is regulated by sheer stress (A), or wnt5a stimulation (B), respectively. *P<0.05.

FIGS. 4A-I. Summarized data showing that tube formation of human Schlemm's canal cells were inhibited by the intervention of anti-ROR1 antibody (A), ROR1 small molecule inhibitor, DB03208 in 0.07% ethanol (B), FZD2 siRNA (C), anti-FZD2 antibody (D), anti-FZD5 antibody (E), CAMK2D siRNA (F), PLCB1 siRNA (CI), PPP3RI siRNA (II), or NFATC3 siRNA (I) in vitro, respectively. isotype control, vehicle control of ethanol (0.07%), or scrambled siRNA were used as negative controls for the antibody, DB03208 small molecule inhibitor, and siRNA treatment assays, respectively. *P<0.05.

FIGS. 5A-B. (A) ROR1 small molecule inhibitor. (B) ROR1 antibody inhibitor.

FIGS. 6A-B. (A) FZD5 antibody inhibitor. (B) FZD2 antibody inhibitor.

### DESCRIPTION OF PARTICULAR EMBODIMENTS OF THE INVENTION

The examples and embodiments described herein are for illustrative purposes and various modifications or changes in light thereof will be apparent to persons skilled in the art and are to be included within this invention. Those skilled in the art will recognize a variety of noncritical parameters that could be changed or modified to yield essentially similar results. The invention may exclude or be practiced in the absence of any compound, component, element or step which is not disclosed are required herein. Unless contraindicated or noted otherwise, in these descriptions and throughout this specification, the terms “a” and “an” mean one or more. All publications, patents, and patent applications cited herein, including citations therein, are hereby incorporated by reference in their entirety for all purposes.

The disclosed Wnt5a receptor/effector inhibition methods can be genetic manipulation, and/or administrations of small interfering RNAs (siRNAs), antibodies, small molecules, etc., many of which are commercially available from sources like Applied Biological Materials Inc. (ABM, Richmond BC), Life Technologies (ThermoFisher Scientific), Sigma-Aldrich, etc. The methods can be used alone to lower intraocular pressure and to prevent or treat glaucoma, and/or in combination with other therapeutic approaches, such as eye drops, medications, laser, implanted devices, and surgery, etc. to prevent or treat glaucoma.

Prototypical Examples

Wnt5a Identification and Targeting

We disclosed Wnt5a identification and targeting in WO2019/040311.

Wnt5a is expressed on human primary SC cells in culture and mouse SC in vivo. Wnt5a expression is regulated with sheer stress change, as analyzed by quantitative real-time PCR assay. We also demonstrate that Wnt5a expression in human SC cells can be down-regulated by Wnt5a-specific siRNA, which affects SC cell functions as well. In SC-specific Wnt5a gene conditional knockout mice IOP elevation induced in a glaucoma model is significantly reduced compared to control littermates. No significant difference was found in baseline IOP between the knockout mice and control littermates. Compared with the control littermates that had IOP elevation at all time-points studied, Wnt5a knockout mice only showed elevated IOP at the early (within 24 hours) but not later time points, indicating an unsustainable IOP increase with Wnt5a intervention. We also demonstrate that wnt5a intervention is effective in protecting retinal nerve fiber layer and increasing SC permeability, a target for enhancement of aqueous movement through the conventional outflow system to manage ocular hypertension (e.g. Tam et al., Scientific Reports 7:40717, DOI: 10.1038/srep40717). These experiments demonstrate Wnt5a is an effective therapeutic target for glaucoma management. These results are further demonstrated by selective inhibition of Wnt5a by CRISPR gene editing employing the methods of Huang, et al. (Nature Communications, 2017; 8 (1) DOI: 10.1038/s41467-017-00140-3).

We next developed experimental protocols to demonstrate efficacy of Wnt5a siRNA inhibitor treatments to reduce IOP. For these protocols Wnt5a specific siRNA was obtained commercially (human WNTSA siRNA, Life Technologies; Anastas, et al. J. Clin. Investig. 2014, 124, 2877-2890). In one protocol subconjuctival injection of siRNA is performed as described by Yuen et al. (2014, Invest Ophthalmol Vis Sci. 2014;55:3320-3327). Mice are randomly selected to receive subconjunctival injection of 5 uL (0.2 lg/uL) siRNA or control twice a week for 2 weeks. In a second protocol intracameral injection of siRNA is performed as described by Tam et al. (2017, Scientific Reports 7, 40717). Mice are anaesthetized by intra-peritoneal injection, and pupils are dilated. A pulled blunt-ended micro-glass needle is first used to puncture the cornea to withdraw aqueous humour Immediately after puncture, a pulled blunt-ended micro-glass needle attached to a 10 μl syringe is inserted through the puncture, and 1.5 μl of PBS containing 1 μg siRNA is administered into the anterior chamber. Contralateral eyes receive an identical injection of 1.5 μl containing the same concentration of scrambled siRNA. These experiments demonstrate that Wnt5a-specific inhibitor siRNA delivered locally by either subconjuctival injection or intracameral injection is an effective therapy for pathogenic IOP.

To assess the effect of siRNA delivered by eye drops on IOP, we developed an additional protocol based on the methods of Martinez et al. (Mol Ther. 2014 January; 22(1):81-91), wherein New Zealand White rabbits receive a topical administration of either 20 nmol/day of siRNA or phosphate-buffered saline (PBS) over a period of 4 consecutive days. Treated eyes present a significant IOP decrease when compared with the vehicle-treated group. The effect of the siRNA on IOP is detectable 2 days after the first administration and values remains below basal levels until ˜2 days after the last administration. We also adapted an oral water overloading model in New Zealand White Rabbits to evaluate the IOP-lowering effect of Wnt5a siRNA in pathologic conditions like observed in glaucoma. Initially four different doses of siRNA (10 nmol, 20 nmol, 40 nmol, and 60 nmol/eye/day) are administered a total of three times: 48, 24, and 2 hours before hypertension induction. All treatments are applied in both eyes and IOP measured before hypertension induction and every 20 minutes up to 120 minutes after oral overloading. Analysis of the results shows that the Wnt5a siRNA provides significant protection against the rise of IOP at all doses tested.

To confirm the efficacy and specificity of Wnt5a siRNA on IOP, a larger group of animals is treated with a dose of 40 nmol/eye/day over a period of 4 consecutive days; on the fourth day, ocular hypertension is induced by water loading. Control results demonstrate that water loading caused an increase in IOP during the first hour after hypertension induction in animals treated with PBS. Analysis performed by comparing IOP values at each time point indicate that treatment with siRNA significantly reduced ΔIOP values within the first hour compared with PBS-treated animals. The effect is specific since treatment with a scrambled sequence siRNA has no effect on IOP.

We next developed experimental protocols to demonstrate efficacy of Wnt5a specific antibody inhibitor treatments to reduce IOP. These protocols employ two different antibodies: anti-human WNTSA antibody produced in rabbit purified immunoglobulin, buffered aqueous solution (Sigma-Aldrich SAB1411396), and an anti-human WNTSA monoclonal antibody produced in mouse clone 6F2, ascites fluid (Sigma-Aldrich SAB5300183), although other Wnt5a antibodies can be used, e.g. Hanaki et al., Mol Cancer Ther 11(2) February 2012; He et al. Oncogene. 2005, 24 (18): 3054-3058. Using both the mouse and rabbit models (supra), these experiments demonstrate that Wnt5a-specific antibody inhibitor delivered locally by eye drops is an effective therapy for pathogenic IOP.

In an exemplary model system intraocular hypertension was induced in the right eye (OD) of wildtype normal mice and Wnt5a neutralizing antibodies were administered to assess their therapeutic effects on IOP and other parameters of glaucoma including corneal edema, retinal ganglion cell (RGC) death, and RNFL thinning Compared to the control group where IOP was significantly elevated in the right eyes of the mice, IOP in Wnt5a antibody treated eyes was significantly lower and maintained at the baseline level. Wnt5a intervention reduced corneal edema, as measured by central corneal thickness in vivo by OCT. Increased corneal thickness was observed in the control group after IOP was increased, but not in Wnt5a antibody treated eyes. Wnt5a intervention reduced RGC death as well as RNFL thinning in the treated eyes. These were detected by immunostaining and OCT, respectively. These results confirmed that local Wnt5a antibody intervention significantly lowers IOP and protects the cornea and retina in a mouse model of glaucoma.

We next designed experimental protocols to demonstrate efficacy of Wnt5a specific antagonist peptide and small molecule inhibitor treatments to reduce IOP. These protocols employ a t-butyloxycarbonyl-modified Wnt5a-derived hexapeptide (Box5) that functions as a potent antagonist of Wnt5a (Jenei, et la., PNAS USA, 106 (46), 19473-8), and 6,7-dihydro -10alpha-hydroxy radicicol, a potent WNT-5A expression inhibitor with relatively low toxicity and excellent stability (Shinonaga et al. Bioorg Med Chem. 2009 Jul. 1; 17(13):4622-35). Again using both the mouse and rabbit models (supra), these experiments demonstrate that Wnt5a-specific modified peptide inhibitor and small molecule inhibitor of Wnt5a expression, delivered locally by eye drops are effective therapies for pathogenic IOP.

Downstream Effector Identification and Targeting

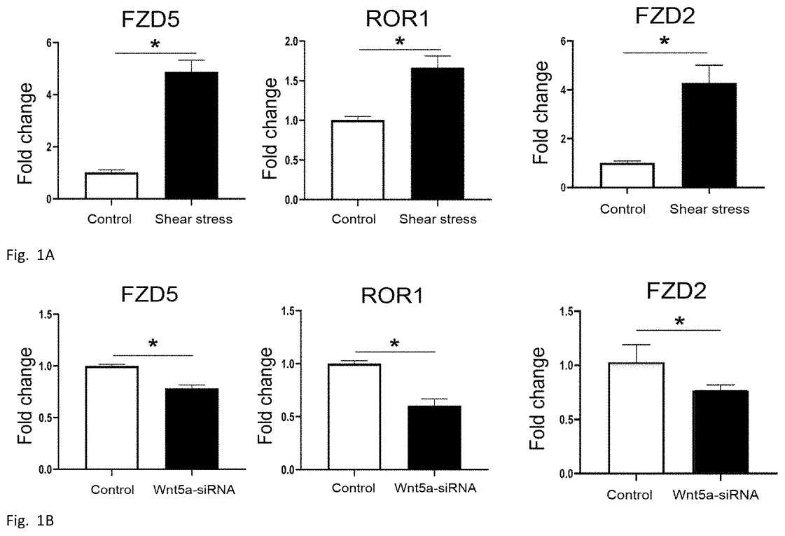

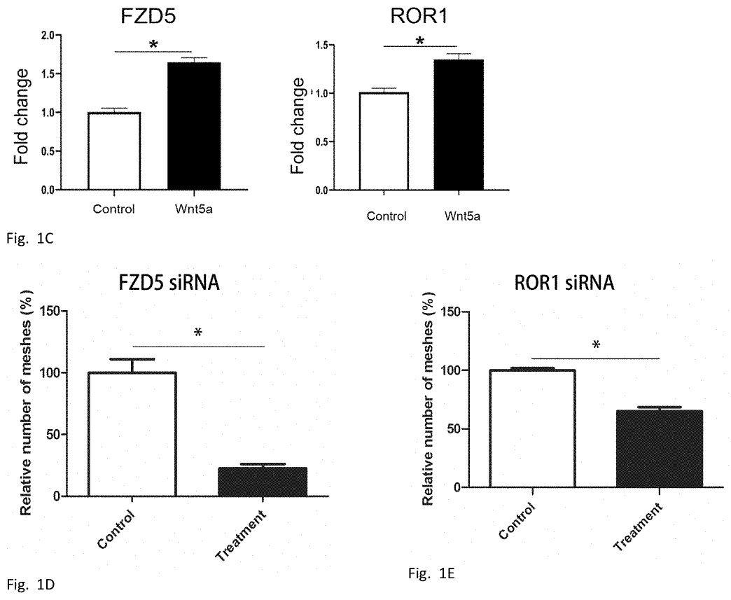

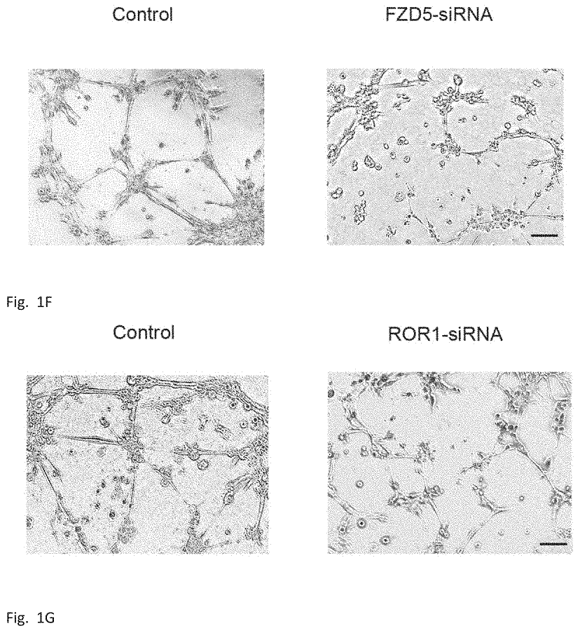

We next demonstrated that FZD5 (frizzled-5), FZD2 and ROR1 (Receptor Tyrosine Kinase Like Orphan Receptor 1), are expressed on SC, their expression is regulated in response to sheer stress change, Wnt5a stimulation or intervention. Moreover, their modulation can regulate SC function, such as tube formation. In particular, in SC cells cultured under pressure (or increased sheer stress), we observed increase of Wnt5a, and Fzd5, Fzd2, RoR1, when Wnt5a is down regulated, Fzd5, Fzd2 and ROR1 are downregulated as well, and when we stimulated human SC cells with Wnt5a, Fzd5, Fzd2 and ROR1 increased correspondingly. See, FIG. 1.

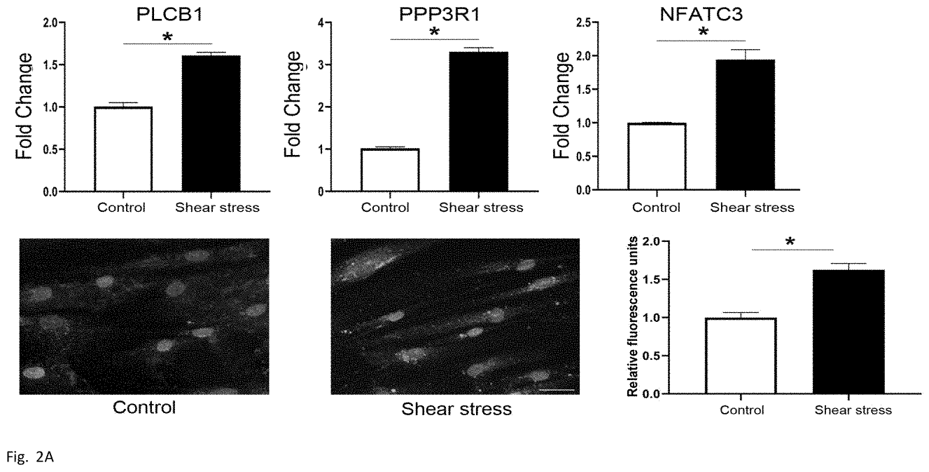

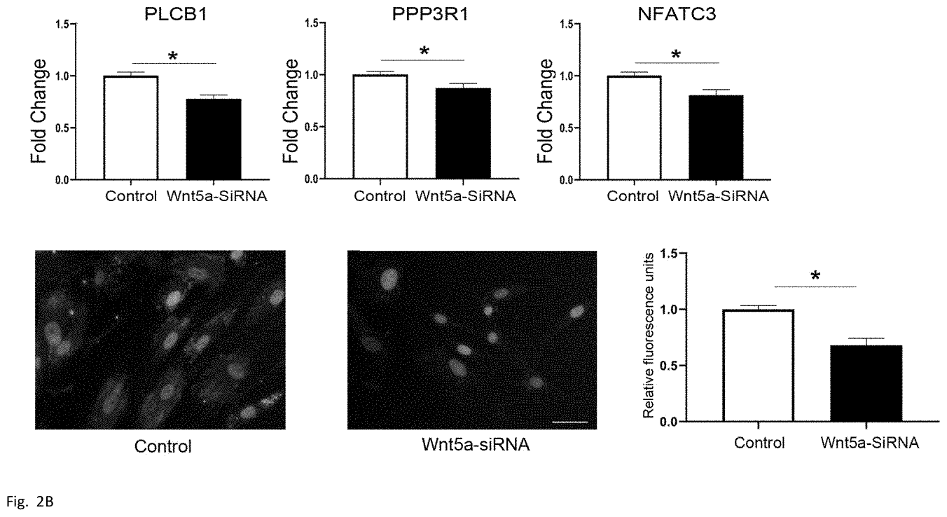

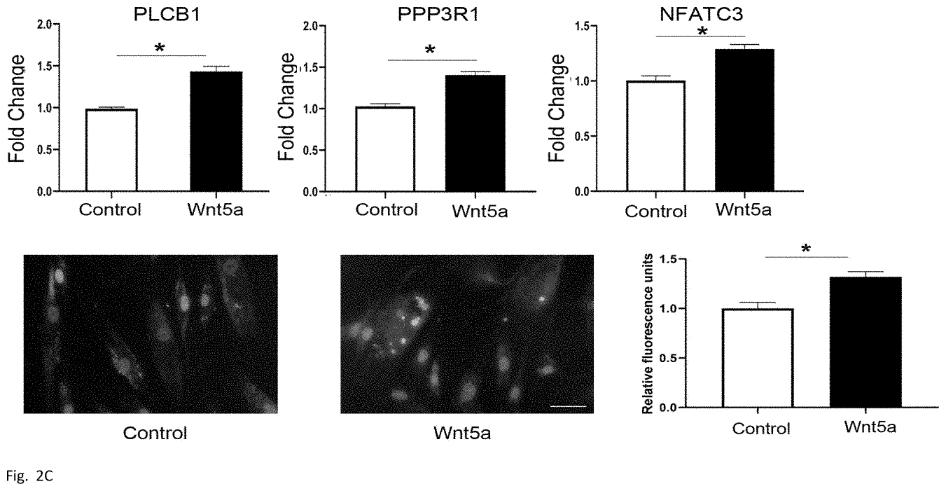

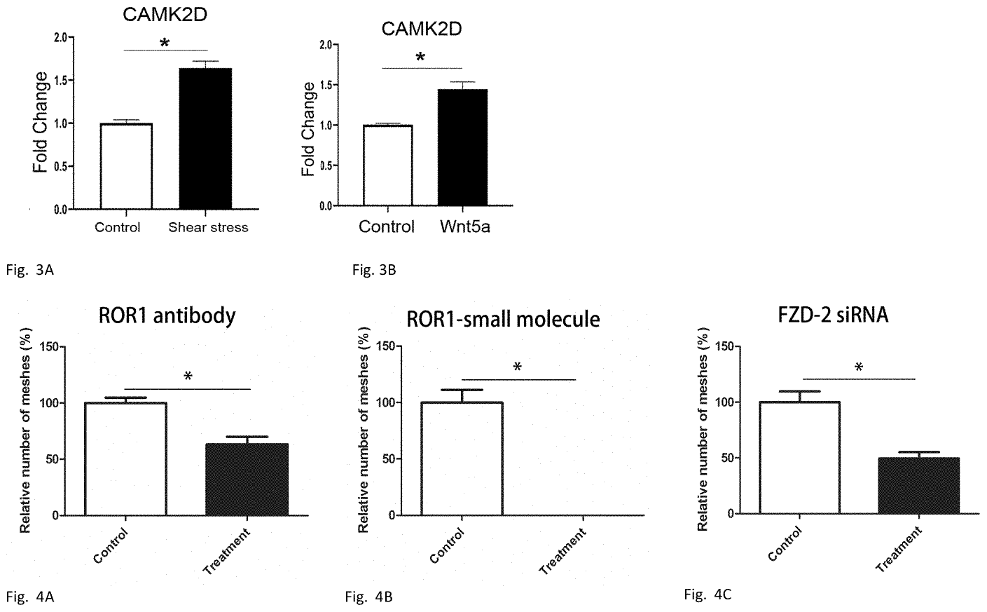

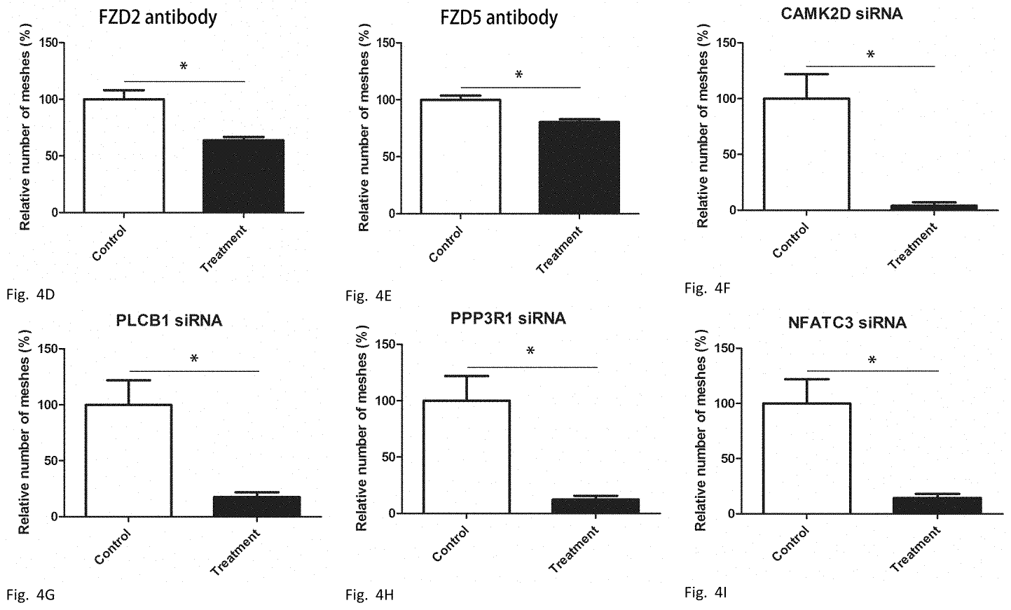

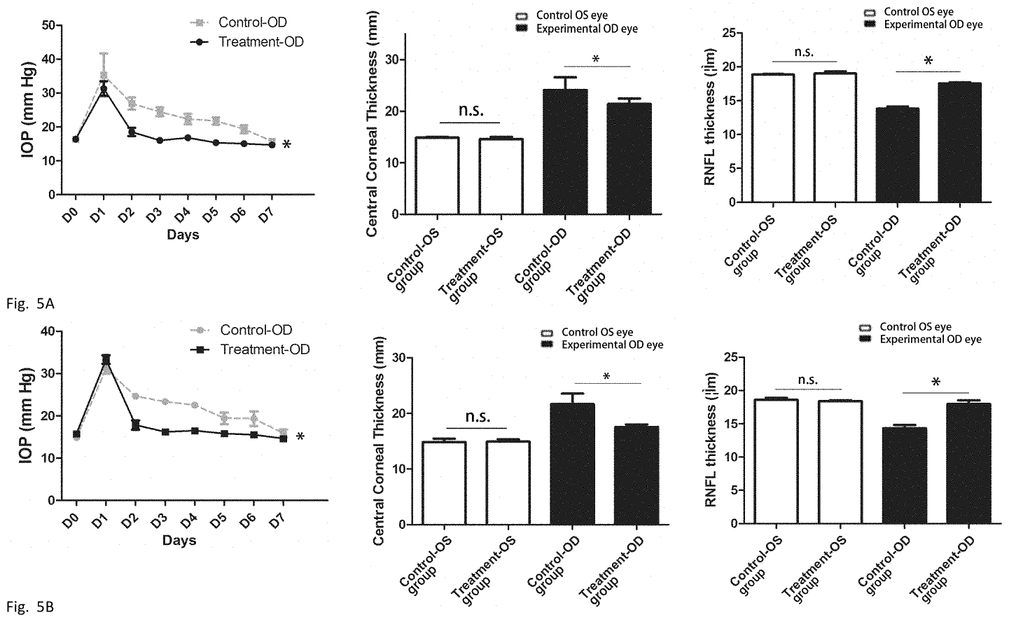

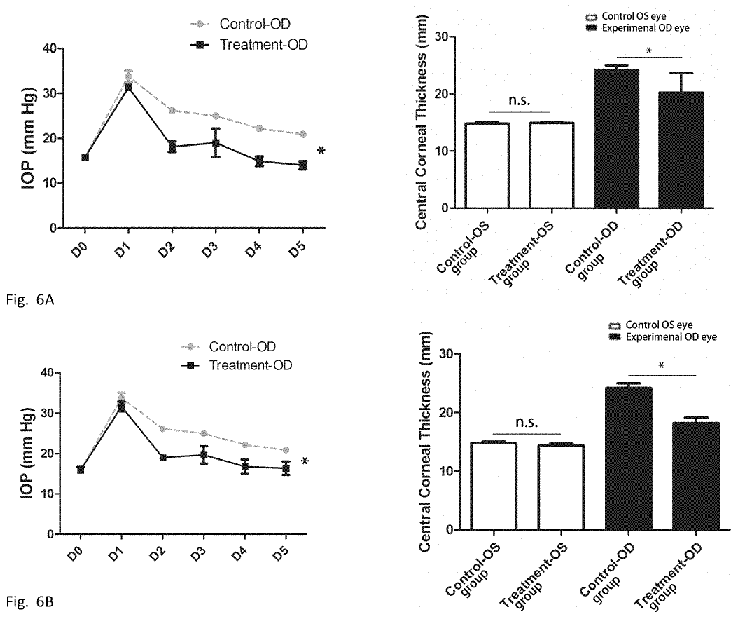

Additionally, we demonstrated that calcium signaling is involved in SC function, and several related molecules, such as PLCB1 (phospholipase C, beta 1), PPP3R1 (Protein Phosphatase 3 Regulatory Subunit B, Alpha), NFATC3 (Nuclear Factor of Activated T Cells 3), CAMK2D (Calcium/Calmodulin Dependent Protein Kinase II Delta), are regulated in response to sheer stress change, Wnt5a stimulation or intervention. See, FIGS. 2 and 3.

We disclose that these Wnt receptors (i.e. FZD5, FZD2, ROR1) and related molecules of calcium signaling pathways (i.e. PLCB1, PPP3R1, NFATC3, CAMK2D) provide targets to modulate SC function and treat glaucoma.

We next developed experimental protocols to demonstrate efficacy of Wnt5a receptor siRNA inhibitor treatments to reduce IOP. For these protocols FZD5, FZD2 and ROR1 specific siRNA was obtained commercially (e.g. ThermoFisher Scientific). In one protocol subconjuctival injection of siRNA is performed as described by Yuen et al. (2014, Invest Ophthalmol Vis Sci. 2014; 55:3320-3327). Mice are randomly selected to receive subconjunctival injection of 5 uL (0.2 lg/uL) FZD5 siRNA, FZD2 siRNA, or ROR1 siRNA or control twice a week for 2 weeks. In a second protocol intracameral injection of FZD5 siRNA, FZD2 siRNA or ROR1 siRNA is performed as described by Tam et al. (2017, Scientific Reports 7, 40717). Mice are anaesthetized by intra-peritoneal injection, and pupils are dilated. A pulled blunt-ended micro-glass needle is first used to puncture the cornea to withdraw aqueous humour. Immediately after puncture, a pulled blunt-ended micro-glass needle attached to a 10 μl syringe is inserted through the puncture, and 1.5 μl of PBS containing 1 μg siRNA is administered into the anterior chamber. Contralateral eyes receive an identical injection of 1.5 μl containing the same concentration of scrambled siRNA. These experiments demonstrate that FZD5, FZD2 and ROR1-specific inhibitor siRNA delivered locally by either subconjuctival injection or intracameral injection is an effective therapy for pathogenic IOP.

To assess the effect of siRNA delivered by eye drops on IOP, we developed an additional protocol based on the methods of Martinez et al. (Mol Ther. 2014 January; 22(1):81-91), wherein New Zealand White rabbits receive a topical administration of either 20 nmol/day of FZD5 siRNA or FZD2 siRNA or ROR1 siRNA or phosphate-buffered saline (PBS) over a period of 4 consecutive days. Treated eyes present a significant IOP decrease when compared with the vehicle-treated group. The effect of the FZD5 siRNA, FZD2 siRNA and ROR1 siRNA on IOP is detectable 2 days after the first administration and values remains below basal levels until 2 days after the last administration. We also adapted an oral water overloading model in New Zealand White Rabbits to evaluate the IOP-lowering effect of FZD5 siRNA, FZD2 siRNA and ROR1 siRNA in pathologic conditions like observed in glaucoma. Initially four different doses of each siRNA (10 nmol, 20 nmol, 40 nmol, and 60 nmol/eye/day) are administered a total of three times: 48, 24, and 2 hours before hypertension induction. All treatments are applied in both eyes and IOP measured before hypertension induction and every 20 minutes up to 120 minutes after oral overloading. Analysis of the results shows that the FZD5 siRNA, FZD2 siRNA and ROR1 siRNA provide significant protection against the rise of IOP at all doses tested.

To confirm the efficacy and specificity of FZD5 siRNA, FZD2 siRNA and ROR1 siRNA on IOP, a larger group of animals is treated with a dose of 40 nmol/eye/day over a period of 4 consecutive days; on the fourth day, ocular hypertension is induced by water loading. Control results demonstrate that water loading caused an increase in IOP during the first hour after hypertension induction in animals treated with PBS. Analysis performed by comparing IOP values at each time point indicate that treatment with FZD5 siRNA, FZD2 siRNA or ROR1 siRNA significantly reduced ΔIOP values within the first hour compared with PBS-treated animals. The effect is specific since treatment with a scrambled sequence siRNA has no effect on IOP.

We next developed experimental protocols to demonstrate efficacy of FZD5, FZD2 and ROR1 specific antibody inhibitor treatments to reduce IOP. These protocols employed OMP18R5 (a humanized monoclonal antibody that binds FZD5), Abcam ab52565 (a monoclonal antibody that binds FZD2), and cirmtuzumab (a humanized IgG1 anti-ROR1 monoclonal antibody), though an anti-human FZD5, FZD2 siRNA and ROR1 polyclonal and monoclonal antibodies, are commercially available from multiple sources, e.g. ThermoFisher Scientific, Abcam, SigmaAldrich, etc.); see also U.S. Pat. No. 9,573,998 for antibodies against human FZD5. Using both the mouse and rabbit models (supra), these experiments demonstrate that FZD5, FZD2 siRNA and ROR1-specific antibody inhibitor delivered locally by eye drops is an effective therapy for pathogenic IOP.

In an exemplary model system intraocular hypertension was induced in the right eye (OD) of wildtype normal mice and FZD5, FZD2 or ROR1 neutralizing antibodies are administered to assess their therapeutic effects on IOP and other parameters of glaucoma including corneal edema, retinal ganglion cell (RGC) death, and RNFL thinning Compared to the control group where IOP is significantly elevated in the right eyes of the mice, IOP in FZD5, FZD2 and ROR1 antibody treated eyes is significantly lower. FZD5, FZD2 and ROR1 intervention reduces corneal edema, as measured by central corneal thickness in vivo by OCT. Increased corneal thickness was observed in the control group after IOP was increased, but not in FZD5, FZD2 and ROR1 antibody treated eyes. FZD5, FZD2 and ROR1 intervention reduced RGC death as well as RNFL thinning in the treated eyes. These are detectable by immunostaining and/or OCT, respectively. These results confirmed that local FZD5, FZD2 and ROR1 antibody intervention significantly lowers IOP and protects the cornea and retina in a mouse model of glaucoma.

We next designed experimental protocols to demonstrate efficacy of FZD5, FZD2 and ROR1 specific antagonist peptides and small molecule inhibitor treatments to reduce IOP. These protocols employ a mutant FZD5 fragment that function as a potent antagonists (e.g. Liu et al., Hum Mol Genet. 2016 Apr. 1; 25(7): 1382-1391 and an oral small molecule inhibitor of ROR1 (KAN0439834; Hojjat-Farsangi et al., Leukemia 32, p2291-2295, 2018). Again using both the mouse and rabbit models (supra), these experiments demonstrate that FZD5, FZD2 and ROR1-specific modified peptide inhibitor and small molecule inhibitors delivered locally by eye drops are effective therapies for pathogenic IOP.

In exemplary models vivo data were obtained using well-established mouse model of glaucoma with laser-induced occlusion of episcleral veins (Zhang L, et al. Establishment and Characterization of an Acute Model of Ocular Hypertension by Laser-Induced Occlusion of Episcleral Veins. Invest Ophthalmol Vis Sci. 2017 Aug. 1; 58(10):3879-3886). Intraocular hypertension is introduced in the right eyes (OD) of the mice with the left eyes (OS) as controls. In these examples, the inhibitors or their controls were delivered daily to the right eyes via local administration of subconjunctival injection, starting from Day 1 after the induction of intraocular hypertension in the right eyes of the mice. Left eyes were used as controls. Central corneal thickness and RNFL were measured on Day 3 or Day 7 by in vivo OCT, respectively. All in vitro data were collected from human Schlemm's cells in culture.

ROR1 Inhibitors

1) Cirmtuzumab

2) KAN0439834, ; Hojjat-Farsang et al., Leukemia. 2018 October; 32(10):2291-2295. doi: 10.1038/s41375-018-0113-1; see also class of related inhibitors: 2-phenyl-3H-imidazo[4,5-b]pyridines (US2018/0002329; U.S. Pat. No. 10,550,113), see below, and incorporated by reference herein.

3) ROR1 siRNAs, Thermofisher

4) ROR1 antibody, R&D Systems, Cat # AF2000

5) ROR1 small molecule, Beta-1,2,3,4,6-Penta-O-Galloyl-D-Glucopyranose (DB03208), Medkoo Biosciences, Inc. Cat #: 564580

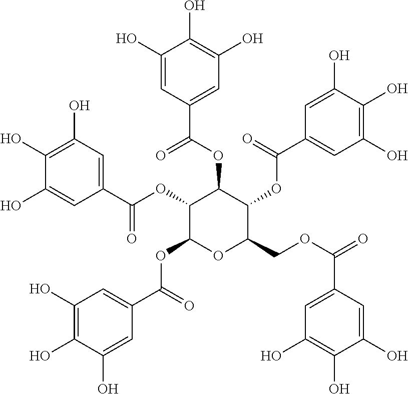 [SMILES: O=C(OC[C@H]1O[C@@H](OC(=O)c2cc(O)c(O)c(O)c2)[C@H](OC(=O)c2cc(O)c(O)c(O)c2)[C@@H](OC(=O)c2cc(O)c(O)c(O)c2)[C@@H]1OC(=O)c1cc(O)c(O)c(O)c1)c1cc(O)c(O)c(O)c1]

6) ROR1 small molecule, 3-O-galloyl-4,6-[(S)-hexahydroxydiphenoyl]-b-d-glucopyranose (strictinin); see: Fultang N, et al. Strictinin, a novel ROR1-inhibitor, represses triple negative breast cancer survival and migration via modulation of PI3K/AKT/GSK3β activity. PLoS One. 2019 May 31; 14(5):e0217789.

7) ROR1 blocking peptide; see: https://www.mybiosource.com/blocking-peptide/ror1/544396

8) (R)-5,7-bis(methoxymethoxy)-2-(4-methoxyphenyl)chroman-4-one (ARI-1), defined as a ROR1 inhibitor; see: Liu X. et al. Novel ROR1 inhibitor ARI-1 suppresses the development of non-small cell lung cancer. Cancer Lett. 2019 August 28; 458:76-85.

9) ROR1-cFab (a chimeric anti-ROR1 Fab antibody); see: Yin Z. et al. Antitumor activity of newly developed monoclonal antibody against ROR1 in ovarian cancer cells. Oncotarget. 2017 Oct. 7; 8(55):94210-94222).

FZD2 Inhibitors

FZD2 siRNAs, Thermofisher

FZD2 antibody, Abcam ab52565

FZD5 Inhibitors

FZD5 siRNAs, Thermofisher

FZD5 antibody, R&D Systems AF1617

CAMK2D Inhibitors

CAMK2D siRNA, Thermofisher

PLCB1 Inhibitors

PLCB1 siRNA, Thermofisher

PPP3R1 Inhibitors

PPP3R1 siRNA, Thermofisher

NFATC3 Inhibitors

1) NFATC3 siRNA, Thermofisher

Recombinant antihuman antibody and variants:

2) Creativebiolabs Recombinant-Anti-Human-NFATC3-Antibody-10188

3) Creativebiolabs Rcombinant-Anti-Human-NFATC3-Antibody-Fab-Fragment-10189

4) Creativebiolabs Rcombinant-Anti-Human-NFATC3-Antibody-scFv-Fragment-10190

TABLE
Human siRNA sequences

| | | | Sequence | Sequence |
|---|---|---|---|---|
| names | Cat# | AssayID | (5′->3′) sense | (5′->3′) antisense |
| Wnt5a | AM16708 | 121437 | GGACCCGCUUAUUUAUAGAtt | UCUAUAAAUAAGCGGGUCCtg |
| | | | (SEQ ID NO: 01) | (SEQ ID NO: 02) |
| ROR1 | AM16708 | 143956 | CCAUCCGCUGGUUCAAAAAtt | UUUUUGAACCAGCGGAUGGtg |
| | | | (SEQ ID NO: 03) | (SEQ ID NO: 04) |
| FZD5 | AM16708 | 139070 | CGUGUAUUCUAUUUUGCGUtt | ACGCAAAAUAGAAUACACGtg |
| | | | (SEQ ID NO: 05) | (SEQ ID NO: 06) |
| FZD2 | AM16708 | 144688 | CGUACUUGGUAGACAUGCAtt | UGCAUGUCUACCAAGUACGtg |
| | | | (SEQ ID NO: 07) | (SEQ ID NO: 08) |
| CAMK2D | AM16708 | 118255 | CCAAAAAGCUUUCUGCUAGtt | CUAGCAGAAAGCUUUUUGGtg |
| | | | (SEQ ID NO: 09) | (SEQ ID NO: 10) |
| PLCB1 | AM16708 | 136825 | CCUCGUGAACAUCUCCCAUtt | AUGGGAGAUGUUCACGAGGtc |
| | | | (SEQ ID NO: 11) | (SEQ ID NO: 12) |
| PPP3R1 | AM16708 | 104462 | GGCUAGGAAAGAGAUUUAAtt | UUAAAUCUCUUUCCUAGCCtt |
| | | | (SEQ ID NO: 13) | (SEQ ID NO: 14) |
| NFATC3 | AM16708 | 105293 | GGUGCACUUUUAUCUUUGCtt | GCAAAGAUAAAAGUGCACCtg |
| | | | (SEQ ID NO: 15) | (SEQ ID NO: 16) |

* ThermoFisher Scientific siRNA gene names

Table. KAN0439834 and related 2-phenyl-3H-imidazo[4,5-b]pyridine ROR1 inhibitors: see US2018/0002329; U.S. Pat. No. 10,550,113.

1. 2-(4-{7-[(1-benzylpiperidin-4-yl)amino]-6-chloro-3H-imidazo[4,5-b]pyridin-2-yl}phenoxy)-N-[2-(dimethylamino)ethyl]acetamide

2. 2-[4-(7-{[(3S)-1-benzylpyrrolidin-3-yl]amino}-6-chloro-3H-imidazo[4,5-b]pyridin-2-yl)phenoxy]-N-[2-(dimethylamino)ethyl]acetamide

3. 2-[4-(6-chloro-7-{[1-(2-phenylethyl)piperidin-4-yl]amino}-3H-imidazo[4,5-b]pyridin-2-yl(phenoxy]-N-[2-(dimethylamino)ethyl]acetamide

4. 2-[4-(7-{[(3S)-1-benzylpyrrolidin-3-yl]amino}-6-chloro-3H-imidazo[4,5-b]pyridin-2-yl)phenoxy]-N-[2-(dimethylamino)-1,1-dimethylethyl]acetamide

5. 2-(4-{7-[(1-benzylpiperidin-4-yl)amino]-6-chloro-3H-imidazo[4,5-b]pyridin-2-yl}phenoxy)-N-[2-(dimethylamino)-1,1-dimethylethyl]acetamide

6. 2-[4-(6-chloro-7-{[1-(4-fluorobenzyl)piperidin-4-yl]amino}-3H-imidazo[4,5-b]pyridin-2-yl(phenoxy]-N-[2-(dimethylamino)-1,1-dimethylethyl]acetamide

7. 2-(4-{7-[(1-benzylpiperidin-3-yl)amino]-6-chloro-3H-imidazo[4,5-b]pyridin-2-yl}phenoxy)-N-[2-(dimethylamino)-1,1-dimethylethyl]acetamide

8. 2-{4-[6-chloro-7-({1-[(3-methyl-2-thienyl)methyl]piperidin-4-yl}amino)-3H -imidazo[4,5-b]pyridin-2-yl]phenoxy}-N-[2-(dimethylamino)-1,1-dimethylethyl]acetamide

9. 2-[4-(6-chloro-7-{[1-(3-methylbenzyl)piperidin-4-yl]amino}-3H-imidazo[4,5-b]pyridin-2-yl(phenoxy]-N-[2-(dimethylamino)ethyl]acetamide

10. 2-[4-(6-chloro-7-{[1-(4-methylbenzyl)piperidin-4-yl]amino}-3H-imidazo[4,5-b]pyridin-2-yl(phenoxy]-N-[2-(dimethylamino)ethyl]acetamide

11. 2-(4-{7-[(1-benzylpiperidin-4-yl)amino]-6-chloro-3H-imidazo[4,5-b]pyridin-2-yl}phenoxy)-N-methylacetamide

12. 2-[4-(7-{[(3S)-1-benzylpyrrolidin-3-yl]amino}-6-chloro-3H-imidazo[4,5-b]pyridin-2-yl)phenoxy]-N-methylacetamide

13. 2-[4-(7-{[1-(1,3-benzodioxol-5-ylmethyl)piperidin-4-yl]amino}-6-chloro-3H -imidazo[4,5-b]pyridin-2-yl)phenoxy]-N-[2-(dimethylamino)ethyl]acetamide

14. 2-[4-(6-chloro-7-{[1-(1,3-thiazol-2-ylmethyl)piperidin-4-yl]amino}-3H -imidazo[4,5-b]pyridin-2-yl)phenoxy]-N-[2-(dimethylamino)ethyl]acetamide

15. 2-[4-(6-chloro-7-{[1-(thiophen-3-ylmethyl)piperidin-4-yl]amino}-3H-imidazo[4,5-b]pyridin-2-yl)phenoxy]-N-[2-(dimethylamino)ethyl]acetamide

16. 2-[4-(7-{[(1-benzylpiperidin-4-yl)methyl]amino}-6-chloro-3H-imidazo[4,5-b]pyridin-2-yl(phenoxy]-N-[2-(dimethylamino)ethyl]acetamide

17. 2-(4-{7-[(1-benzylpiperidin-4-yl)(methyl)amino]-6-chloro-3H-imidazo[4,5-b]pyridin-2-yl}phenoxy)-N-[2-(diethylamino)ethyl]acetamide

18. 2-(4-{7-[(1-benzylpiperidin-4-yl)(methyl)amino]-6-chloro-3H-imidazo[4,5-b]pyridin-2-yl}phenoxy)-N-[2-(dimethylamino)-2-methylpropyl]acetamide

19. -(4-{7-[(1-benzylpiperidin-4-yl)(methyl)amino]-6-chloro-3H-imidazo[4,5-b]pyridin-2-yl}phenoxy)-N-methylacetamide

20. 2-(4-{7-[(1-benzylpiperidin-4-yl)(methyl)amino]-6-chloro-3H-imidazo[4,5-b]pyridin-2-yl}phenoxy)-N-[2-(dimeethylamino)ethyl]acetamide

21. 2-(4-{7-[(1-benzylpiperidin-4-yl)(methyl)amino]-6-chloro-3H-imidazo[4,5-b]pyridin-2-yl}phenoxy)-N-[2-(dimethylamino)-1-methylethyl]acetamide

22. 2-[4-(6-chloro-7-{[1-(4-chlorobenzyl)piperidin-4-yl]amino}-3H-imidazo[4,5-b]pyridin-2-yl(phenoxy]-N-[2-(dimethylamino)ethyl]acetamide

23. 2-(4-{7-[(1-benzylpiperidin-4-yl)amino]-6-chloro-3H-imidazo[4,5-b]pyridin-2-yl}phenoxy)-N-[2-(dimethylamino)-2-methylpropyl]acetamide

24. 2-[4-(7-{[(3R)-1-benzylpyrrolidin-3-yl]amino}-6-chloro-3H-imidazo[4,5-b]pyridin-2-yl)phenoxy]-N-[2-(dimethylamino)-2-methylpropyl]acetamide

25. 2-[4-(7-{[(3S)-1-benzylpyrrolidin-3-yl]amino}-6-chloro-3H-imidazo[4,5-b]pyridin-2-yl)phenoxy]-N-[2-(dimethylamino)-2-methylpropyl]acetamide

26. 2-[4-(6-chloro-7-{[1-(4-fluorobenzyl)piperidin-4-yl]amino}-3H-imidazo[4,5-b]pyridin-2-yl(phenoxy]-N-[2-(dimethylamino)-2-methylpropyl]acetamide

27. 2-[4-(6-chloro-7-{[1-(4-methylbenzyl)piperidin-4-yl]amino}-3H-imidazo[4,5-b]pyridin-2-yl(phenoxy]-N-[2-(dimethylamino)-2-methylpropyl]acetamide

28. 2-[4-(6-chloro-7-{[1-(3-methylbenzyl)piperidin-4-yl]amino}-3H-imidazo[4,5-b]pyridin-2-yl(phenoxy]-N-[2-(dimethylamino)-2-methylpropyl]acetamide

29. 2-{4-[6-chloro-7-({1-[(5-methylfuran-2-yl)methyl]piperidin-4-yl}amino)-3H -imidazo[4,5-b]pyridin-2-yl]phenoxy}-N-[2-(dimethylamino)ethyl]acetamide

30. 2-[4-(7-{[(3R)-1-benzylpyrrolidin-3-yl]amino}-6-chloro-3H-imidazo[4,5-b]pyridin-2-yl)phenoxy]-N-[2-(dimethylamino)ethyl]acetamide

31. 2-[4-(7-{[(1-benzylpiperidin-4-yl)methyl]amino}-6-chloro-3H-imidazo[4,5-b]pyridin-2-yl(phenoxy]-N-[2-(dimeethylamino)-2-methylpropyl]acetamide

32. 2-[4-(6-chloro-7-{[(3S)-1-(3,4-difluorobenzyl)pyrrolidin-3-yl]amino}-3H -imidazo[4,5-b]pyridin-2-yl)phenoxy]-N-[2-(dimethylamino)ethyl]acetamide

33. 2-[4-(6-chloro-7-{[(3S)-1-(4-fluorobenzyl)pyrrolidin-3-yl]amino}-3H-imidazo[4,5-b]pyridin-2-yl)phenoxy]-N-[2-(dimethylamino)ethyl]acetamide

34. 2-[4-(6-chloro-7-{[(3S)-1-(3,4-difluorobenzyl)pyrrolidin-3-yl]amino}-3H -imidazo[4,5-b]pyridin-2-yl)phenoxy]-N-methylacetamide

35. 2-[4-(6-chloro-7-{[(3S)-1-(4-fluorobenzyl)pyrrolidin-3-yl]amino}-3H-imidazo[4,5-b]pyridin-2-yl)phenoxy]-N-methylacetamide

36. 2-[4-(6-chloro-7-{[(3R)-1-(4-fluorobenzyl)pyrrolidin-3-yl]amino}-3H -imidazo[4,5-b]pyridin-2-yl)phenoxy]-N-[2-(dimethylamino)ethyl]acetamide

37. 2-[4-(6-chloro-7-{[(3R)-1-(4-fluorobenzyl)pyrrolidin-3-yl]amino}-3H -imidazo[4,5-b]pyridin-2-yl)phenoxy]-N-methylacetamide

38. 2-[4-(7-{[(3R)-1-benzylpyrrolidin-3-yl]amino}-6-chloro-3H-imidazo[4,5-b]pyridin-2-yl)phenoxy]-N-methylacetamide

39. 2-[4-(6-chloro-7-{[1-(thiophen-3-ylmethyl)piperidin-4-yl]amino}-3H-imidazo[4,5-b]pyridin-2-yl)phenoxy]-N-meethylacetamide

40. 2-[4-(6-chloro-7-{[1-(3-methylbenzyl)piperidin-4-yl]amino}-3H-imidazo[4,5-b]pyridin-2-yl(phenoxy]-N-methylacetamide

41. 2-[4-(6-chloro-7-{[(3S)-1-(2-phenylethyl)pyrrolidin-3-yl]amino}-3H-imidazo[4,5-b]pyridin-2-yl)phenoxy]-N-methylacetamide

42. 2-{4-[6-chloro-7-({1-[(3-methyl-2-thienyl)methyl]piperidin-4-yl}amino)-3H -imidazo[4,5-b]pyridin-2-yl]phenoxy}-N-methylacetamide

43. 2-[4-(6-chloro-7-{[(3S)-1-(4-methoxybenzyl)pyrrolidin-3-yl]amino}-3H -imidazo[4,5-b]pyridin-2-yl)phenoxy]-N-methylacetamide

44. 2-[4-(6-chloro-7-{[1-(4-methoxybenzyl)piperidin-4-yl]amino}-3H-imidazo[4,5-b]pyridin-2-yl(phenoxy]-N-methylacetamide

45. 2-[4-(7-{[(1,3-benzodioxol-5-ylmethyl)piperidin-4-yl]amino}-6-chloro-3H -imidazo[4,5-b]pyridin-2-yl)phenoxy]-N-methylacetamide

46. 2-{4-[6-chloro-7-({1-[(5-methylfuran-2-yl)methyl]piperidin-4-yl}amino)-3H -imidazo[4,5-b]pyridin-2-yl]phenoxy}-N-methylacetamide

47. 2-[4-(6-chloro-7-{[(3S)-1-(thiophen-3-ylmethyl)pyrrolidin-3-yl]amino}-3H -imidazo[4,5-b]pyridin-2-yl)phenoxy]-N-methylacetamide

48. 2-[4-(6-chloro-7-{[1-(furan-3-ylmethyl)piperidin-4-yl]amino}-3H-imidazo[4,5-b]pyridin-2-yl(phenoxy]-N-methylacetamide

49. 2-[4-(7-{[(3S)-1-(1,3-benzodioxol-5-ylmethyl)pyrrolidin-3-yl]amino}-6-chloro-3H -imidazo[4,5-b]pyridin-2-yl)phenoxy]-N-methylacetamide

50. 2-[4-(6-chloro-7-{[(3S)-1-(1,3-thiazol-2-ylmethyl)pyrrolidin-3-yl]amino}-3H -imidazo[4,5-b]pyridin-2-yl)phenoxy]-N-methylacetamide

51. 2-{4-[6-chloro-7-({(3S)-1-[(3-methyl-2-thienyl)methyl]pyrrolidin-3-yl}amino)-3H -imidazo[4,5-b]pyridin-2-yl]phenoxy}-N-methylacetamide

52. 2-{4-[6-chloro-7-({(3S)-1-[4-(trifluoromethyl)benzyl]pyrrolidin-3-yl}amino)-3H -imidazo[4,5-b]pyridin-2-yl]phenoxy}-N-methylacetamide

53. 2-[4-(6-chloro-7-{[(3S)-1-(3-methylbenzyl)pyrrolidin-3-yl]amino}-3H -imidazo[4,5-b]pyridin-2-yl)phenoxy]-N-methylacetamide

54. 2-[4-(6-chloro-7-{[(3S)-1-(4-methylbenzyl)pyrrolidin-3-yl]amino}-3H -imidazo[4,5-b]pyridin-2-yl)phenoxy]-N-methylacetamide

55. 2-[4-(6-chloro-7-{[(3S)-1-(2-thienylmethyl)pyrrolidin-3-yl]amino}-3H -imidazo[4,5-b]pyridin-2-yl)phenoxy]-N-methylacetamide

56. 2-(4-{6-chloro-7-[(1-cyclohexylpiperidin-4-yl)amino]-3H-imidazo[4,5-b]pyridin-2-yl}phenoxy)-N-methylacetamide

57. 2-[4-(6-chloro-7-{[(3S)-1-(3-methoxybenzyl)pyrrolidin-3-yl]amino}-3H -imidazo[4,5-b]pyridin-2-yl)phenoxy]-N-methylacetamide

58. 2-[4-(6-chloro-7-}[(3S)-1-(2-methoxybenzyl)pyrrolidin-3-yl]amino}-3H -imidazo[4,5-b]pyridin-2-yl)phenoxy]-N-methylacetamide

59. 2-[4-(6-chloro-7-{[(3S)-1-(2-methylbenzyl)pyrrolidin-3-yl]amino}-3H -imidazo[4,5-b]pyridin-2-yl)phenoxy]-N-methylacetamide

60. 2-[4-(6-chloro-7-{[1-(2,4-dimethoxybenzyl)piperidin-4-yl]amino}-3H -imidazo[4,5-b]pyridin-2-yl)phenoxy]-N-methylacetamide

61. 2-[4-(6-chloro-7-{[1-(2-methoxybenzyl)piperidin-4-yl]amino}-3H-imidazo[4,5-b]pyridin-2-yl(phenoxy]-N-methylacetamide

62. 2-[4-(6-chloro-7-{[1-(3-methoxybenzyl)piperidin-4-yl]amino}-3H-imidazo[4,5-b]pyridin-2-yl(phenoxy]-N-methylacetamide

63. 2-(4-{7-[(1-benzylpiperidin-4-yl)amino]-6-chloro-3H-imidazo[4,5-b]pyridin-2-yl}phenoxy)-N,N-dimethylacetamide

64. 2-[4-(7-{[(3S)-1-benzylpyrrolidin-3-yl]amino}-6-chloro-3H-imidazo[4,5-b]pyridin-2-yl)phenoxy]-N,N-dimethylacetamide

65. 2-[4-(6-chloro-7-{[1-(2,3-dihydro-1,4-benzodioxin-6-ylmethyl)piperidin-4-yl]amino}-3H-imidazo[4,5-b]pyridin-2-yl)phenoxy]-N-methylacetamide

66. 2-[4-(6-chloro-7-{[1-(cyclohexylmethyl)piperidin-4-yl]amino}-3H-imidazo[4,5-b]pyridin-2-yl)phenoxy]-N-methylacetamide

67. 2-[4-(6-chloro-7-{[1-(2,2-dimethylpropyl)piperidin-4-yl]amino}-3H-imidazo[4,5-b]pyridin-2-yl)phenoxy]-N-methylacetamide

68. 2-[4-(6-chloro-7-1[1-(3-hydroxybenzyl)piperidin-4-yl]amino}-3H-imidazo[4,5-b]pyridin-2-yl(phenoxy]-N-methylacetamide

69. 2-{4-[6-chloro-7-({1-[4-(difluoromethoxy)benzyl]piperidin-4-yl}amino)-3H -imidazo[4,5-b]pyridin-2-yl]phenoxy}-N-methylacetamide

70. 2-[4-(6-chloro-7-{[1-(4-methoxy-3-methylbenzyl)piperidin-4-yl]amino}-3H -imidazo[4,5-b]pyridin-2-yl)phenoxy]-N-methylacetamide

71. 2-[4-(6-chloro-7-{[1-(pyridin-4-ylmethyl)piperidin-4-yl]amino}-3H-imidazo[4,5-b]pyridin-2-yl)phenoxy]-N-methylacetamide

72. 2-[4-(6-chloro-7-{[1-(pyridin-3-ylmethyl)piperidin-4-yl]amino}-3H-imidazo[4,5-b]pyridin-2-yl)phenoxy]-N-methylacetamide

73. 2-{4-[6-chloro-7-({1-[(1-methyl-1H-pyrrol-2-yl)methyl]piperidin-4-yl}amino)-3H -imidazo[4,5-b]pyridin-2-yl]phenoxy}-N-methylacetamide

74. 2-{4-[6-chloro-7-({1-[(6-methylpyridin-2-yl)methyl]piperidin-4-yl}amino)-3H -imidazo[4,5-b]pyridin-2-yl]phenoxy}-N-methylacetamide

75. 2-[4-(7-{[1-(4-acetamidobenzyl)piperidin-4-yl]amino}-6-chloro-3H-imidazo[4,5-b]pyridin-2-yl)phenoxy]-N-methylacetamide

76. 2-[4-(6-chloro-7-{[1-(1,3-thiazol-2-ylmethyl)piperidin-4-yl]amino}-3H -imidazo[4,5-b]pyridin-2-yl)phenoxy]-N-methylacetamide

77. 2-[4-(6-chloro-7-{[1-(4-ethoxybenzyl)piperidin-4-yl]amino}-3H-imidazo[4,5-b]pyridin-2-yl)phenoxy]-N-methylacetamide

78. 2-[4-(6-chloro-7-{[1-(4-isopropoxybenzyl)piperidin-4-yl]amino}-3H-imidazo[4,5-b]pyridin-2-yl)phenoxy]-N-methylacetamide

79. 2-[4-(7-{[(1-benzylpiperidin-4-yl)methyl]amino}-6-chloro-3H-imidazo[4,5-b]pyridin-2-yl)phenoxy]-N-methylacetamide

80. 2-[4-(6-chloro-7-{[1-(4-methoxy-3,5-dimethylbenzyl)piperidin-4-yl]amino}-3H -imidazo[4,5-b]pyridin-2-yl)phenoxy]-N-methylacetamide

81. 2-[4-(6-chloro-7-{[1-(4-chlorobenzyl)piperidin-4-yl]amino}-3H-imidazo[4,5-b]pyridin-2-yl)phenoxy]-N-methylacetamide

82. 2-[4-(6-chloro-7-{[1-(4-methylbeiizyl)piperidin-4-yl]amino}-3H-imidazo[4,5-b]pyridin-2-yl)phenoxy]-N-methylacetamide

83. 2-[4-(6-chloro-7-{[1-(4-cyanobenzyl)piperidin-4-yl]amino}-3H-imidazo[4,5-b]pyridin-2-yl)phenoxy]-N-methylacetamide

84. 2-[4-(6-chloro-7-{[1-(3-cyanobenzyl)piperidin-4-yl]amino}-3H-imidazo[4,5-b]pyridin-2-yl)phenoxy ]-N-methylacetamide

85. 2-[4-(6-chloro-7-{[1-(4-hydroxybenzyl)piperidin-4-yl]amino}-3H-imidazo[4,5-b]pyridin-2-yl)phenoxy]-N-methylacetamide

86. 2-{4-[6-chloro-7-(piperidin-4-ylamino)-3H-imidazo[4,5-b]pyridin-2-yl]phenoxy}-N-methylacetamide

87. 2-[4-(6-chloro-7-{[1-(4-fluorobenzyl)piperidin-4-yl]amino}-3H-imidazo[4,5-b]pyridin-2-yl(phenoxy]-N-methylacetamide

88. 2-[4-(6-chloro-7-{[1-(3,4-difluorobenzyl)piperidin-4-yl]amino}-3H-imidazo[4,5-b]pyridin-2-yl)phenoxy]-N-methylacetamide

89. 2-{4-[6-chloro-7-({1-[4-(dimethylamino)benzyl]piperidin-4-yl}amino)-3H -imidazo[4,5-b]pyridin-2-yl]phenoxy}-N-methylacetamide

90. 2-{4-[6-chloro-7-({1-[4-(methylsulfonyl)benzyl]piperidin-4-yl}amino)-3H -imidazo[4,5-b]pyridin-2-yl]phenoxy}-N-methylacetamide

91. 2-[4-(6-chloro-7-{[1-(2,3-dihydro-1-benzofuran-5-ylmethyl)piperidin-4-yl]amino}-3H-imidazo[4,5-b]pyridin-2-yl)phenoxy]-N-methylacetamide

92. 2-(4-{6-chloro-7-[(1-methylpiperidin-4-yl)amino]-3H-imidazo[4,5-b]pyridin-2-yl}phenoxy)-N-methylacetamide

93. 2-[4-(6-chloro-7-{[1-(2-thienylmethyl)piperidin-4-yl]amino}-3H-imidazo[4,5-b]pyridin-2-yl(phenoxy]-N-methylacetamide

94. 2-[4-(6-chloro-7-{[1-(2-phenylethyl)piperidin-4-yl]amino}-3H-imidazo[4,5-b]pyridin-2-yl(phenoxy]-N-methylacetamide

95. 2-{4-[6-chloro-7-({1-[2-(4-methoxyphenyl)ethyl]piperidin-4-yl}amino)-3H -imidazo[4,5-b]pyridin-2-yl]phenoxy}-N-methylacetamide

96. 2-[4-(6-chloro-7-{[1-(2-phenoxyethyl)piperidin-4-yl]amino}-3H-imidazo[4,5-b]pyridin-2-yl(phenoxy]-N-methylacetamide

97. 2-[4-(6-chloro-7-{[1-(3,4-dimethoxybenzyl)piperidin-4-yl]amino}-3H -imidazo[4,5-b]pyridin-2-yl)phenoxy]-N-methylacetamide

98. 2-[4-(6-chloro-7-{[1-(4-hydroxy-3-methoxybenzyl)piperidin-4-yl]amino}-3H -imidazo[4,5-b]pyridin-2-yl)phenoxy]-N-methylacetamide

99. 2-{4-[6-chloro-7-({1-[4-(1H-1,2,4-triazol-1-yl)benzyl]piperidin-4-yl}amino)-3H -imidazo[4,5-b]pyridin-2-]phenoxy}-N-methylacetamide

100. 2-{4-[6-chloro-7-({1-[4-(methylthio)benzyl]piperidin-4-yl}amino)-3H -imidazo[4,5-b]pyridin-2-yl]phenoxy}-N-methylacetamide

101. 2-[4-(6-chloro-7-{[1-(4-methoxybenzyl)piperidin-4-yl]amino}-3H-imidazo[4,5-b]pyridin-2-yl(phenoxy]-N-(2-hydroxyethyl)acetamide

102. 2-[4-(6-chloro-7-{[1-(4-methoxybenzyl)piperidin-4-yl]amino}-3H-imidazo[4,5-b]pyridin-2-yl(phenoxy]-N-[2-(dimethylamino)ethyl]acetamide

103. 2-[4-(6-chloro-7-{[1-(4-methoxybenzyl)pipendin-4-yl]amino}-3H-imidazo[4,5-b]pyridin-2-yl(phenoxy]-N-[2-(dimethylamino)-2-methylpropyl]acetamide

104. 2-[4-(6-chloro-7-{[1-(4-methoxybenzyl)piperidin-4-yl]amino}-3H-imidazo[4,5-b]pyridin-2-yl(phenoxy]-N-isopropylacetamide

105. 2-[4-(6-chloro-7-{[1-(4-methoxybenzyl)piperidin-4-yl]amino}-3H-imidazo[4,5-b]pyridin-2-yl(phenoxy]-N-(2-isopropoxyethyl)acetamide

106. 3-[4-(6-chloro-7-{[1-(4-methoxybenzyl)piperidin-4-yl]amino}-3H-imidazo[4,5-b]pyridin-2-yl(phenyl]-N-methylpropanamide

107. 3-[4-(6-chloro-7-{[1-(4-methoxybenzyl)piperidin-4-yl]amino}-3H-imidazo[4,5-b]pyridin-2-yl(phenyl]-N-[2-(dimethylamino)ethyl]propanamide

108. 3-[4-(6-chloro-7-{[1-(4-methoxybenzyl)piperidin-4-yl]amino}-3H-imidazo[4,5-b]pyridin-2-yl(phenyl]-N-methoxypropanamide

109. 2-(4-{6-chloro-7-[(1-ethylpiperidin-4-yl)amino]-3H-imidazo[4,5-b]pyridin-2-yl}phenoxy)-N-methylacetamide

110. 2-[4-(6-chloro-7-{[1-(1-methylethyl)piperidin-4-yl]amino}-3H-imidazo[4,5-b]pyridin-2-yl(phenoxy]-N-methylacetamide

111. 2-[4-(6-bromo-7-{[1-(4-methoxybenzyl)piperidin-4-yl]amino}-3H-imidazo[4,5-b]pyridin-2-yl(phenoxy]-N-methylacetamide

112. 2-[4-(6-bromo-7-{[1-(2,3-dihydro-1-benzofuran-5-ylmethyl)piperidin-4-yl]amino}-3H-imidazo[4,5-b]pyridin-2-yl)phenoxy]-{V-methylacetamide

113. 2-[4-(6-bromo-7-{[1-(thiophen-2-ylmethyl)piperidin-4-yl]amino}-3H -imidazo[4,5-/?]pyridin-2-yl)phenoxy]-N-methylacetamide

114. 2-(4-{6-bromo-7-[(1-methylpiperidin-4-yl)amino]-3H-imidazo[4,5-b]pyridin-2-yl}phenoxy)-N-methylacetamide

115. 2-[4-(6-bromo-7-{[(3S)-1-(2-methoxybenzyl)pyrrolidin-3-yl]amino}-3H -imidazo[4,5-b]pyridin-2-yl)phenoxy]-N-methylacetamide

116. 2-[3-(6-chloro-7-{[1-(4-methoxybenzyl)piperidin-4-yl]amino}-3H-imidazo[4,5-b]pyridin-2-yl(phenoxy]-N-methylacetamide

117. 2-(3-{6-chloro-7-[(1-methylpiperidin-4-yl)amino]-3H-imidazo[4,5-b]pyridin-2-yl}phenoxy)-N-methylacetamide

118. 2-[3-(6-chloro-7-{[1-(2,3-dihydro-1-benzofuran-5-ylmethyl)piperidin-4-yl]amino}-3H-imidazo[4,5-b]pyridin-2-yl)phenoxy]-N-methylacetamide

119. 2-[3-(6-chloro-7-{[1-(thiophen-2-ylmethyl)piperidin-4-yl]amino}-3H -imidazo[4,5-b]pyridin-2-yl)phenoxy]-N-methylacetamide

120. 2-[3-(7-{[1-(1,3-benzodioxol-5-ylmethyl)piperidin-4-yl]amino}-6-chloro-3H -imidazo[4,5-b]pyridin-2-yl)phenoxy]-N-methylacetamide

121. 2-[3-(6-chloro-7-{[1-(2-phenoxyethyl)piperidin-4-yl]amino}-3H-imidazo[4,5-b]pyridin-2-yl)phenoxy]-N-methylacetamide

122. 2-[3-(6-chloro-7-{[(3S)-1-(2-methoxybenzyl)pyrrolidin-3-yl]amino}-3H -imidazo[4,5-b]pyridin-2-yl)phenoxy]-N-methylacetamide

123. 2-[3-(7-{[(3S)-1-benzylpyrrolidin-3-yl]amino}-6-chloro-3H-imidazo[4,5-b]pyridin-2-yl)phenoxy]-N-methylacetamide

124. 2-(4-{6-chloro-7-[(1-hexylpiperidin-4-yl)amino]-3H-imidazo[4,5-b]pyridin-2-yl}phenoxy)-N-methylacetamide

125. 2-[4-(6-chloro-7-{[1-(2-methylpropyl)piperidin-4-yl]amino)-3H-imidazo[4,5-b]pyridin-2-yl)phenoxy]-N-methylacetamide

126. 2-(4-{6-chloro-7-[(1-propylpiperidin-4-yl)amino]-3H-imidazo[4,5-b]pyridin-2-yl}phenoxy)-N-methylacetamide

127. 2-(4-{6-chloro-7-[(1,2,2,6,6-pentamethylpiperidin-4-yl)amino]-3H-imidazo[4,5-b]pyridin-2-yl}phenoxy)-N-methylacetamide

128. 2-{3-[6-chloro-7-({1-[4-(1H-1,2,4-triazol-1-yl)benzyl]piperidin-4-yl}amino)-3H -imidazo[4,5-b]pyridin-2-yl]phenoxy}-N-methylacetamide

129. 2-[4-(6-chloro-7-{[(3S)-1-methylpyrrolidin-3-yl]amino}-3H-imidazo[4,5-b]pyridin-2-yl)phenoxy]-N-methylacetamide

130. 2-[3-(6-chloro-7-{[1-(3-thienylmethyl)piperidin-4-yl]amino}-3H-imidazo[4,5-b]pyridin-2-yl)phenoxy]-N-methylacetamide

131. 2-[3-(6-chloro-7-{[1-(3-hydroxybenzyl)piperidin-4-yl]amino}-3H-imidazo[4,5-b]pyridin-2-yl)phenoxy]-N-methylacetamide

132. 2-[4-(6-chloro-7-{[(3S)-1-ethylpyrrolidin-3-yl]amino}-3H-imidazo[4,5-b]pyridin-2-yl)phenoxy]-N-methylacetamide

133. 2-[4-(6-chloro-7-{[(3S)-1-propylpyrrolidin-3-yl]amino}-3H-imidazo[4,5-b]pyridin-2-yl)phenoxy]-N-methylacetamide

134. 2-[4-(6-chloro-7-{[(3S)-1-(1-methylethyl)pyrrolidin-3-yl]amino}-3H -imidazo[4,5-b]pyridin-2-yl)phenoxy]-N-methylacetamide

135. 2-(4-(6-chloro-7-[(1-methylpiperidin-4-yl)amino]-3H-imidazo[4,5-b]pyridin-2-yl}phenoxy)-N-ethylacetamide

136. 2-(4-{6-chloro-7-[(1-methylpiperidin-4-yl)amino]-3H-imidazo[4,5-b]pyridin-2-yl}phenoxy)-N-isopropylacetamide

137. 2-(4-{6-chloro-7-[(1-methylpiperidin-4-yl)amino]-3H-imidazo[4,5-b]pyridin-2-yl}phenoxy)-N-cyclopentylacetamide

138. 2-(4-{6-bromo-7-[(1-ethylpiperidin-4-yl)amino]-3H-imidazo[4,5-b]pyridin-2-yl}phenoxy)-N-methylacetamide

139. 2-(4-{6-chloro-7-[(1-methylpiperidin-4-yl)amino]-3H-imidazo[4,5-b]pyridin-2-yl}phenoxy)-N-methoxyacetamide

140. 2-(4-{6-bromo-7-[(1-propylpiperidin-4-yl)amino]-3H-imidazo[4,5-b]pyridin-2-yl}phenoxy)-N-methylacetamide

141. 2-[4-(6-bromo-7-{[1-(1-methylethyl)piperidin-4-yl]amino}-3H-imidazo[4,5-b]pyridin-2-yl(phenoxy]-N-methylacetamide

142. 2-(4-{6-chloro-7-[(1-methylpiperidin-4-yl)amino]-3H-imidazo[4,5-b]pyridin-2-yl}phenoxy)-N-(2-isopropoxyethyl)acetamide

143. 2-(4-{6-chloro-7-[(1-methylpiperidin-4-yl)amino]-3H-imidazo[4,5-b]pyridin-2-yl}phenoxy)-N-[2-(dimethylamino)ethyl]acetamide

144. 2-[4-(6-bromo-7-{[(3S)-1-methylpyrrolidin-3-yl]amino}-3H-imidazo[4,5-b]pyridin-2-yl(phenoxy]-N-methylacetamide

145. 2-(4-{6-chloro-7-[(1-methylpiperidin-4-yl)amino]-3H-imidazo[4,5-b]pyridin-2-yl}phenoxy)-N-(2-cyclohexylethyl)acetamide

146. 2-(4-{6-chloro-7-[(1-methylpiperidin-4-yl)amino]-3H-imidazo[4,5-b]pyridin-2-yl}phenoxy)-N-(cyclohexylmethyl)acetamide

147. 2-(4-{6-chloro-7-[(1-methylpiperidin-4-yl)amino]-3H-imidazo[4,5-b]pyridin-2-yl}phenoxy)-N-[2-(tetrahydro-2H-pyran-4-yl)ethyl]acetamide

148. 2-(4-{6-chloro-7-[(1-methylpiperidin-4-yl)amino]-3H-imidazo[4,5-b]pyridin-2-yl}phenoxy)-N-(tetrahydro-2H-pyran-4-ylmethyl)acetamide

149. 2-(4-{6-chloro-7-[(1-methylpiperidin-4-yl)amino]-3H-imidazo[4,5-b]pyridin-2-yl}phenoxy)-N-[2-(1-methylpiperidin-4-yl)ethyl]acetamide bis(trifluoroacetate)

150. 2-(4-{6-chloro-7-[(1-methylpiperidin-4-yl)amino]-3H-imidazo[4,5-b]pyridin-2-yl}phenoxy)-N-[(1-methylpiperidin-4-yl)methyl]acetamide

151. 2-(4-{6-chloro-7-[(1-methylpiperidin-4-yl)amino]-3H-imidazo[4,5-b]pyridin-2-yl}phenoxy)-N-(piperidin-4-ylmethyl)acetamide

152. 2-(4-{6-chloro-7-[(1-methylpiperidin-4-yl)amino]-3H-imidazo[4,5-b]pyridin-2-yl}phenoxy)-N-(2-morpholin-4-ylethyl)acetamide

153. 2-(4-{6-chloro-7-[(1-methylpiperidin-4-yl)amino]-3H-imidazo[4,5-b]pyridin-2-yl}phenoxy)-N-(3-morpholin-4-ylpropyl)acetamide

154. 2-[4-(6-chloro-7-{[1-(2-methoxyethyl)piperidin-4-yl]amino}-3H-imidazo[4,5-b]pyridin-2-yl(phenoxy]-N-methylacetamide

155. 2-(4-{6-chloro-7-[(1-methylpiperidin-4-yl)amino]-3H-imidazo[4,5-b]pyridin-2-yl}phenoxy)-N-(2-piperidin-4-ylethyl)acetamide

156. 2-(4-{6-chloro-7-[(1-methylpiperidin-4-yl)amino]-3H-imidazo[4,5-b]pyridin-2-yl}-3-methylphenoxy)-N-methylacetamide

157. 2-[4-(6-chloro-7-{[1-(4-methoxybenzyl)piperidin-4-yl]amino}-3H-imidazo[4,5-b]pyridin-2-yl)-3-methylphenoxy]-N-methylacetamide

158. 2-(4-{6-bromo-7-[(1-ethylpiperidin-4-yl)amino]-3H-imidazo[4,5-b]pyridin-2-yl}-3-methylphenoxy)-N-methylacetamide

159. 2-(4-{6-bromo-7-[(1-methylpiperidin-4-yl)amino]-3H-imidazo[4,5-b]pyridin-2-yl}-3-methylphenoxy)-N-methylacetamide

160. 2-[4-(6-bromo-7-{[1-(4-methoxybenzyl)piperidin-4-yl]amino}-3H-imidazo[4,5-b]pyridin-2-yl)-3-methylphenoxy]-N-methylacetamide

161. 2-[4-(6-chloro-7-{[1-(4-methoxybenzyl)piperidin-4-yl]amino}-3H-imidazo[4,5-b]pyridin-2-yl)-2-methylphenoxy]-N-methylacetamide

162. 2-(4-{6-chloro-7-[(1-methylpiperidin-4-yl)amino]-3H-imidazo[4,5-b]pyridin-2-yl}-2-methylphenoxy)-N-methylacetamide

163. 2-(4-{6-bromo-7-[(1-methylpiperidin-4-yl)amino]-3H-imidazo[4,5-b]pyridin-2-yl}-2-methylphenoxy)-N-methylacetamide

164. 2-(4-{6-bromo-7-[(1-ethylpiperidin-4-yl)amino]-3H-imidazo[4,5-b]pyridin-2-yl}-2-methylphenoxy)-N-methylacetamide

165. 2-(4-{6-bromo-7-[(1-propylpiperidin-4-yl)amino]-3H-imidazo[4,5-b]pyridin-2-yl}-2-methylphenoxy)-N-methylacetamide

166. 2-[4-(6-chloro-7-{[(3S)-1-(1-methylethyl)pyrrolidin-3-yl]amino}-3H -imidazo[4,5-b]pyridin-2-yl)-2-methylphenoxy]-N-methylacetamide

167. 2-(4-{6-bromo-7-[(1-methylpiperidin-4-yl)amino]-3H-imidazo[4,5-b]pyridin-2-yl}-2-methoxyphenoxy)-N-methylacetamide

168. 2-(4-{6-chloro-7-[(1-methylpiperidin-4-yl)amino]-3H-imidazo[4,5-b]pyridin-2-yl}-2-methoxyphenoxy)-N-methylacetamide

169. 2-[4-(6-chloro-7-{[1-(4-methoxybenzyl)piperidin-4-yl]amino}-3H-imidazo[4,5-b]pyridin-2-yl)-2-methoxyphenoxy]-N-methylacetamide

170. 2-(4-{6-bromo-7-[(1-propylpiperidin-4-yl)amino]-3H-imidazo[4,5-b]pyridin-2-yl}-2-methoxyphenoxy)-N-methylacetamide

171. 2-[4-(6-chloro-7-{[(3S)-1-(1-methylethyl)pyrrolidin-3-yl]amino}-3H -imidazo[4,5-b]pyridin-2-yl)-2-methoxyphenoxy]N-methylacetamide

172. 2-[4-(6-chloro-7-{[1-(2,3-dihydro-1-benzofuran-5-ylmethyl)piperidin-4-yl]amino}-3H-imidazo[4,5-b]pyridin-2-yl)-3-methylphenoxy]-N-methylacetamide

173. 2-(4-{6-chloro-7-[(1-ethylpiperidin-4-yl)amino]-3H-imidazo[4,5-b]pyridin-2-yl}-3-methylphenoxy)-N-methylacetamide

174. 2-[4-(6-chloro-7-{[1-(thiophen-3-ylmethyl)piperidin-4-yl]amino}-3H -imidazo[4,5-b]pyridin-2-yl)-3-methylphenoxy]-N-methylacetamide

175. 2-[4-(6-chloro-7-{[(3S)-1-(2-methoxybenzyl)pyrrolidin-3-yl]amino}-3H -imidazo[4,5-b]pyridin-2-yl)-2-methylphenoxy]-N-methylacetamide

176. 2-[4-(6-chloro-7-{[1-(thiophen-3-ylmethyl)piperidin-4-yl]amino}-3H -imidazo[4,5-b]pyridin-2-yl)-2-methylphenoxy]-N-methylacetamide

177. 2-(4-{6-chloro-7-[(1-ethylpiperidin-4-yl)amino]-3H-imidazo[4,5-b]pyridin-2-yl}-2-methylphenoxy)-N-methylacetamide

178. 2-[4-(6-chloro-7-{[1-(2-methoxyethyl)piperidin-4-yl]amino}-3H-imidazo[4,5-b]pyridin-2-yl)-2-methylphenoxy]-N-methylacetamide

179. 2-(4-{6-bromo-7-[(1-methylpiperidin-4-yl)amino]-3H-imidazo[4,5-b]pyridin-2-yl}phenoxy)-N,2-dimethylpropanamide

180. 2-(4-{6-chloro-7-[(1-methylpiperidin-4-yl)amino]-3H-imidazo[4,5-b]pyridin-2-yl}phenoxy)-N,2-dimethylpropanamide

181. 2-[4-(6-chloro-7-{[1-(4-methoxybenzyl)piperidin-4-yl]amino}-3H-imidazo[4,5-b]pyridin-2-yl(phenoxy]-N,2-dimethylpropanamide

182. 2-(4-{6-bromo-7-[(1-ethylpiperidin-4-yl)amino]-3H-imidazo[4,5-b]pyridin-2-yl}phenoxy)-N,2dimethylpropanamide

183. 2-[4-(6-chloro-7-{[(3S)-1-(1-methylethyl)pyrrolidin-3-yl]amino}-3H -imidazo[4,5-b]pyridin-2-yl)phenoxy]-N,2-dimethylpropanamide

184. 2-(4-{6-bromo-7-[(1-methylpiperidin-4-yl)amino]-3H-imidazo[4,5-b]pyridin-2-yl}-2-fluorophenoxy)-N-methylacetamide

185. 2-(4-{6-chloro-7-[(1-methylpiperidin-4-yl)amino]-3H-imidazo[4,5-b]pyridin-2-yl}-2-fluorophenoxy)-N-methylacetamide

186. 2-[4-(6-chloro-7-{[1-(4-methoxybenzyl)piperidin-4-yl]amino}-3H-imidazo[4,5-b]pyridin-2-yl)-2-fluorophenoxy]-N-methylacetamide

187. 2-(4-{6-bromo-7-[(1-ethylpiperidin-4-yl)amino]-3H-imidazo[4,5-b]pyridin-2-yl}-2-fluorophenoxy)-N-methylacetamide

188. 2-[4-(6-chloro-7-{[(3S)-1-ethylpyrrolidin-3-yl]amino}-3H-imidazo[4,5-b]pyridin-2-yl)-2-fluorophenoxy]-N-methylacetamide

189. 3-(4-{6-bromo-7-[(1-methylpiperidin-4-yl)amino]-3H-imidazo[4,5-b]pyridin-2-yl}phenyl)-N-methylpropanamide

190. 3-(4-{6-chloro-7-[(1-methylpiperidin-4-yl)amino]-3H-imidazo[4,5-b]pyridin-2-yl}phenyl)-N-methylpropanamide

191. 3-[4-(6-bromo-7-{[1-(1-methylethyl)piperidin-4-yl]amino}-3H-imidazo[4,5-b]pyridin-2-yl(phenyl]-N-methylpropanamide

192. 3-(4-{6-bromo-7-[(1-ethylpiperidin-4-yl)amino]-3H-imidazo[4,5-b]pyridin-2-yl}phenyl)-N-methylpropanamide

193. 3-[4(6-chloro-7-{[(3S)-1-ethylpyrrolidin-3-yl{amino}-3H-imidazo[4,5-b]pyridin-2-yl)phenyl]-N-methylpropanamide

194. 2-[4-(6-chloro-7-{[1-(4-methoxybenzyl)piperidin-4-yl]amino}-3H-imidazo[4,5-b]pyridin-2-yl)-2,6-dimethylphenoxy]-N-methylacetamide

195. 2-(4-{6-chloro-7-[(1-methylpiperidin-4-yl)amino]-3H-imidazo[4,5-b]pyridin-2-yl}-2,6-dimethylphenoxy)-N-methylacetamide

196. 2-(4-{6-bromo-7-[(1-methylpiperidin-4-yl)amino]-3H-imidazo[4,5-b]pyridin-2-yl}-2,6-dimethylphenoxy)-N-methylacetamide

197. 2-[4-(6-bromo-7-{[1-(1-methylethyl)piperidin-4-yl]amino}-3H-imidazo[4,5-b]pyridin-2-yl)-2,6-dimethylphenoxy]-N-methylacetamide

198. 2-[4-(6-chloro-7-{[(3S)-1-(1-methylethyl)pyrrolidin-3-yl]amino}-3H -imidazo[4,5-b]pyridin-2-yl)-2,6-dimethylphenoxy]-N-methylacetamide

199. 2-[4-(6-bromo-7-{[1-(thiophen-2-ylmethyl)piperidin-4-yl]amino}-3H -imidazo[4,5-b]pyridin-2-yl)-2,6-dimethylphenoxy]-N-methylacetamide

200. 2-[4-(6-chloro-7-{[(3S)-1-ethylpyrrolidin-3-yl]amino}-3H-imidazo[4,5-b]pyridin-2-yl)-2,6-dimethylphenoxy]-N-methylacetamide

201. 2-[4-(6-chloro-7-{[1-(4-methoxybenzyl)piperidin-4-yl]amino}-3H-imidazo[4,5-b]pyridin-2-yl)-2,5-dimethylphenoxy]-{V-methylacetamide

202. 2-(4-{6-chloro-7-[(1-methylpiperidin-4-yl)amino]-3H-imidazo[4,5-b]pyridin-2-yl}-2,5-dimethylphenoxy)-N-methylacetamide

203. 2-(4-{6-bromo-7-[(1-methylpiperidin-4-yl)amino]-3H-imidazo[4,5-b]pyridin-2-yl}-2,5-dimethylphenoxy)-N-methylacetamide

204. 2-(4-{6-chloro-7-[(1-propylpiperidin-4-yl)amino]-3H-imidazo[4,5-b]pyridin-2-yl}-2,5-dimethylphenoxy)-ALmethylacetamide

205. 2-(4-{6-chloro-7-[(1-ethylpiperidin-4-yl)amino]-3H-imidazo[4,5-b]pyridin-2-yl}-2,5-dimethylphenoxy)-N-methylacetamide

206. 2-(4-{6-chloro-7-[(1-propylpiperidin-4-yl)amino]-3H-imidazo[4,5-b]pyridin-2-yl}-2-methylphenoxy)-N-methylacetamide

207. 2-(4-{6-chloro-7-[(1-ethylpiperidin-4-yl)amino]-3H-imidazo[4,5-b]pyridin-2-yl}-2,6-dimethylphenoxy)-N-methylacetamide

208. 2-(4-{6-chloro-7-[(1-methylpiperidin-4-yl)amino]-3H-imidazo[4,5-b]pyridin-2-yl}phenoxy)-N-(4-methylcyclohexyl)acetamide

209. N-tert-butyl-2-(4-{6-chloro-7-[(1-methylpiperidin-4-yl)amino]-3H-imidazo[4,5-b]pyridin-2-yl}phenoxy)acetamide

210. 2-(4-{6-chloro-7-[(1-methylpiperidin-4-yl)amino]-3H-imidazo[4,5-b]pyridin-2-yl}phenoxy)-N-(1,1-dimethylpropyl)acetamide

211. 2-(4-{6-chloro-7-[(1-methylpiperidin-4-yl)amino]-3H-imidazo[4,5-b]pyridin-2-yl}phenoxy)-N-cyclohexylacetamide

212. 3-(4-{6-chloro-7-[(1-ethylpiperidin-4-yl)amino]-3H-imidazo[4,5-b]pyridin-2-yl}phenyl)-N-methylpropanamide

213. 3-[4-(6-chloro-7-{[1-(1-methylethyl)piperidin-4-yl]amino}-3H-imidazo[4,5-b]pyridin-2-yl(phenyl]-N-methylpropanamide

214. 2-(4-{6-chloro-7-[(1-ethylpiperidin-4-yl)amino]-3H-imidazo[4,5-b]pyridin-2-yl}-2-fluorophenoxy)-N-methylacetamide

215. 2-[4-(6-chloro-7-{[1-(1-methylethyl)piperidin-4-yl]amino}-3H-imidazo[4,5-b]pyridin-2-yl)-2-fluorophenoxy]-N-methylacetamide

216. 2-(4-{6-chloro-7-[(1-ethylpiperidin-4-yl)amino]-3H-imidazo[4,5-b]pyridin-2-yl}phenoxy)-N,2-dimethylpropanamide

217. 2-[4-(6-chloro-7-{[1-(1-methylethyl)piperidin-4-yl]amino}-3H-imidazo[4,5-b]pyridin-2-yl(phenoxy]-N,2-dimethylpropanamide

218. 2-(4-{6-chloro-7-[(1-ethylpiperidin-4-yl)amino]-3H-imidazo[4,5-b]pyridin-2-yl}-2-methoxyphenoxy)-N-methylacetamide

219. 2-[4-(6-chloro-7-{[1-(1-methylethyl)piperidin-4-yl]amino}-3H-imidazo[4,5-b]pyridin-2-yl)-2-methoxyphenoxy]-N-methylacetamide

220. 2-(4-{6-chloro-7-[(1-methylpiperidin-4-yl)amino]-3H-imidazo[4,5-b]pyridin-2-yl}phenoxy)-N-propylacetamide

221. 2-(4-{6-chloro-7-[(1-methylpiperidin-4-yl)amino]-3H-imidazo[4,5-b]pyridin-2-yl}phenoxy)-N-(2-methylpropyl)acetamide

222. 2-(4-{6-chloro-7-[(1-methylpiperidin-4-yl)amino]-3H-imidazo[4,5-b]pyridin-2-yl}phenoxy)-N-(tetrahydrofuran-2-ylmethyl)acetamide

223. 2-(4-(6-chloro-7-[(1-methylpiperidin-4-yl)amino]-3H-imidazo[4,5-b]pyridin-2-yl}phenoxy)-N-[1-(methoxymethyl)propyl]acetamide

224. 2-(4-{6-chloro-7-[(1-methylpiperidin-4-yl)amino]-3H-imidazo[4,5-b]pyridin-2-yl}phenoxy)-N-(2-methoxy-1-methylethyl)acetamide

225. N-benzyl-2-(4-{6-chloro-7-[(1-methylpiperidin-4-yl)amino]-3H-imidazo[4,5-6]pyridin-2-yl}phenoxy)acetamide

226. 2-(4-{6-chloro-7-[(1-methylpiperidin-4-yl)amino]-3H-imidazo[4,5-b]pyridin-2-yl}phenoxy)-N-(1-phenylethyl)acetamide

227. 2-(4-{6-chloro-7-[(1-methylpiperidin-4-yl)amino]-3H-imidazo[4,5-b]pyridin-2-yl}phenoxy)-N-cycloheptylacetamide

228. 2-(4-{6-bromo-7-[(1-methylpiperidin-4-yl)amino]-3H-imidazo[4,5-b]pyridin-2-yl}-2-fluorophenoxy)-N-(1-methylethyl)acetamide

229. 2-(4-{6-chloro-7-[(1-methylpiperidin-4-yl)amino]-3H-imidazo[4,5-b]pyridin-2-yl}-2-fluorophenoxy)-N-(1-methylethyl)acetamide

230. 2-(4-{6-chloro-7-[(1-ethylpiperidin-4-yl)amino]-3H-imidazo[4,5-b]pyridin-2-yl}-2-fluorophenoxy)-N-(1-methylethyl)acetamide

231. 2-[4-(6-chloro-7-{[1-(1-methylethyl)piperidin-4-yl]amino}-3H-imidazo[4,5-b]pyridin-2-yl)-2-fluorophenoxy]-N-(1-methylethyl)acetamide

232. 2-(4-{6-chloro-7-[(1-methylpiperidin-4-yl)amino]-3H-imidazo[4,5-b]pyridin-2-yl}phenyl)-N-methylacetamide

233. 2-[4-(6-chloro-7-{[1-(4-methoxybenzyl)piperidin-4-yl]amino}-3H-imidazo[4,5-b]pyridin-2-yl(phenyl]-N-methylacetamide

234. 2-[4-(6-chloro-7-{[1-(4-methoxybenzyl)piperidin-4-yl]amino}-3H-imidazo[4,5-b]pyridin-2-yl(phenoxy]-N-cyclopentylacetamide

235. 2-[4-(6-chloro-7-{[1-(4-methoxybenzyl)piperidin-4-yl]amino}-3H-imidazo[4,5-b]pyridin-2-yl(phenoxy]-N-(cyclohexylmethyl)acetamide

236. 2-[4-(6-chloro-7-{[1-(4-methoxybenzyl)piperidin-4-yl]amino}-3H-imidazo[4,5-b]pyridin-2-yl(phenoxy]-N-cycloheptylacetamide

237. 2-[4-(6-chloro-7-{[1-(4-methoxybenzyl)piperidin-4-yl]amino}-3H-imidazo[4,5-b]pyridin-2-yl(phenoxy]-N-(2-cyclohexylethyl)acetamide

238. 2-[4-(6-chloro-7-{[1-(4-methoxybenzyl)piperidin-4-yl]amino}-3H-imidazo[4,5-b]pyridin-2-yl(phenoxy]-N-[2-(tetrahydro-2H-pyran-4-yl)ethyl]acetamide

239. 2-(4-{6-chloro-7-[(1-methylpiperidin-4-yl)amino]-3H-imidazo[4,5-b]pyridin-2-yl}phenoxy)-N-(4-methoxybenzyl)acetamide

240. 2-(4-{6-chloro-7-[(1-methylpiperidin-4-yl)amino]-3H-imidazo[4,5-b]pyridin-2-yl}phenoxy)-N-(furan-2-ylmethyl)acetamide

241. 2-(4-(6-chloro-7-[(1-methylpiperidin-4-yl)amino]-3H-imidazo[4,5-b]pyridin-2-yl}phenoxy)-N-(thiophen-2-ylmethyl)acetamide

242. 2-(4-{6-chloro-7-[(1-methylpiperidin-4-yl)amino]-3H-imidazo[4,5-b]pyridin-2-yl}phenoxy)-N-(2-methoxyethyl)acetamide

243. 2-(4-{6-chloro-7-[(1-methylpiperidin-4-yl)amino]-3H-imidazo[4,5-b]pyridin-2-yl}phenoxy)-N-pyridin-4-ylacetamide

244. 2-(4-{6-chloro-7-[(1-ethylpiperidin-4-yl)amino]-3H-imidazo[4,5-b]pyridin-2-yl}phenyl)-N-methylacetamide

245. 2-[4-(6-chloro-7-{[1-(1-methylethyl)piperidin-4-yl]amino}-3H-imidazo[4,5-b]pyridin-2-yl(phenyl]-N-methylacetamide

246. 2-(4-{6-chloro-7-[(1-propylpiperidin-4-yl)amino]-3H-imidazo[4,5-b]pyridin-2-yl}phenyl)-N-methylacetamide

247. 2-(4-{6-bromo-7-[(1-propylpiperidin-4-yl)amino]3H-imidazo[4,5-b]pyridin-2-yl}phenyl)-N-methylacetamide

248. 2-[4-(6-bromo-7-{[1-(1-methylethyl)piperidin-4-yl]amino}-3H-imidazo[4,5-b]pyridin-2-yl(phenyl]-N-methylacetamide

249. 2-(4-{6-bromo-7-[(1-methylpiperidin-4-yl)amino]-3H-imidazo[4,5-b]pyridin-2-yl}phenyl)-N-methylacetamide

250. 2-[4-(6-chloro-7-{[1-(thiophen-2-ylmethyl)piperidin-4-yl]amino}-3H -imidazo[4,5-b]pyridin-2-yl)phenoxy]-N-cyclopentylacetamide

251. 2-[4-(6-chloro-7-{[1-(thiophen-2-ylmethyl)piperidin-4-yl]amino}-3H -imidazo[4,5-b]pyridin-2-yl)phenoxy]-N-cyclohexylacetamide

252. 2-(4-{6-bromo-7-[(1-methylpiperidin-4-yl)amino]-3H-imidazo[4,5-b]pyridin-2-yl}phenoxy)-N-(1-methylethyl)acetamide

253. 2-(4-{6-bromo-7-[(1-methylpiperidin-4-yl)amino]-3H-imidazo[4,5-b]pyridin-2-yl}phenoxy)-N-propylacetamide

254. 2-(4-{6-bromo-7-[(1-methylpiperidin-4-yl)amino]-3H-imidazo[4,5-b]pyridin-2-yl}phenoxy)-N-[1-(methoxymethyl)propyl]acetamide

255. 2-(4-{6-bromo-7-[(1-methylpiperidin-4-yl)amino]-3H-imidazo[4,5-b]pyridin-2-yl}phenoxy)-N-(2-methylpropyl)acetamide

256. 2-(4-{6-bromo-7-[(1-methylpiperidin-4-yl)amino]-3H-imidazo[4,5-b]pyridin-2-yl}phenoxy)-N-tert-butylacetamide

257. 2-(4-{6-bromo-7-[(1-methylpiperidin-4-yl)amino]-3H-imidazo[4,5-b]pyridin-2-yl}phenoxy)-N-(1,1-dimethylpropyl)acetamide

258. 2-(4-{6-bromo-7-[(1-methylpiperidin-4-yl)amino]-3H-imidazo[4,5-b]pyridin-2-yl}phenoxy)-N-cyclohexylacetamide

259. 2-(4-{6-bromo-7-[(1-methylpiperidin-4-yl)amino]-3H-imidazo[4,5-b]pyridin-2-yl}phenoxy)-N-cyclopentyl acetamide

260. 2-(4-{6-bromo-7-[(1-methylpiperidin-4-yl)amino]-3H-imidazo[4,5-b]pyridin-2-yl}phenoxy)-N-ethylacetamide

261. 2-(4-{6-chloro-7-[(1-methylpiperidin-4-yl)amino]-3H-imidazo[4,5-b]pyridin-2-yl}phenoxy)-N-(tetrahydro-2H-thiopyran-4-yl)acetamide

262. 2-(4-{6-chloro-7-[(1-methylpiperidin-4-yl)amino]-3H-imidazo[4,5-b]pyridin-2-yl}phenoxy)-N-(tetrahydro-2H-pyran-4-yl)acetamide

263. 2-(4-{6-chloro-7-[(1-methylpiperidin-4-yl)amino]-3H-imidazo[4,5-b]pyridin-2-yl}phenoxy)-N-(2,2,2-trifluoroethyl)acetamide

264. N-{2-[4-(6-chloro-7-{[1-(4-methoxybenzyl)piperidin-4-yl]amino}-3H -imidazo[4,5-b]pyridin-2-yl)phenoxy]ethyl}acetamide

265. N-[2-(4-{6-chloro-7-[(1-methylpiperidin-4-yl)amino]-3H-imidazo[4,5-b]pyridin-2-yl}phenoxy)ethyl]acetamide

266. N-[2-(4-{6-bromo-7-[(1-methylpiperidin-4-yl)amino]-3H-imidazo[4,5-b]pyridin-2-yl}phenoxy)ethyl]acetamide

267. N-{2-[4-(6-chloro-7-{[1-(1-methylethyl)piperidin-4-yl]amino}-3H-imidazo[4,5-b]pyridin-2-yl)phenoxy]ethyl}acetamide

268. N-[2-(4-{6-chloro-7-[(1-methylpiperidin-4-yl)amino]-3H-imidazo[4,5-b]pyridin-2-yl}phenoxy)ethyl]cyclohexanecarboxamide

269. N-[2-(4-{6-chloro-7-[(1-methylpiperidin-4-yl)amino]-3H-imidazo[4,5-b]pyridin-2-yl}phenoxy)ethyl]-2,2-dimethylpropanamide

270. 2-(4-{6-bromo-7-[(1-methylpiperidin-4-yl)amino]-3H-imidazo[4,5-b]pyridin-2-yl}phenoxy)-N-pyridin-4-ylacetamide

271. 2-[4-(6-chloro-7-{[1-(1-methylethyl)piperidin-4-yl]amino}-3H-imidazo[4,5-b]pyridin-2-yl)phenoxy]-N-cyclohexylacetamide

272. N-[2-(4-{6-chloro-7-[(1-methylpiperidin-4-yl)amino]-3H-imidazo[4,5-b]pyridin-2-yl}phenoxy)ethyl]pyridine-4-carboxamide

273. N-[2-(4-{6-chloro-7-[(1-methylpiperidin-4-yl)amino]-3H-imidazo[4,5-b]pyridin-2-yl}phenoxy)ethyl]pyridine-3-carboxamide

274. N-[2-(4-{6-chloro-7-[(1-methylpiperidin-4-yl)amino]-3H-imidazo[4,5-b]pyridin-2-yl}phenoxy)ethyl]-2-methoxyacetamide

275. N-[2-(4-{6-chloro-7-[(1-methylpiperidin-4-yl)amino]-3H-imidazo[4,5-b]pyridin-2-yl}phenoxy)ethyl]cyclopentanecarboxamide

276. N-[2-(4-{6-chloro-7-[(1-methylpiperidin-4-yl)amino]-3H-imidazo[4,5-b]pyridin-2-yl}phenoxy)ethyl]-2-methylpropanamide

277. N-[2-(4-{6-chloro-7-[(1-methylpiperidin-4-yl)amino]-3H-imidazo[4,5-b]pyridin-2-yl}phenoxy)ethyl]cyclopropanecarboxamide

278. N-[2-(4-(6-chloro-7-[(1-ethylpiperidin-4-yl)amino]-3H-imidazo[4,5-b]pyridin-2-yl}phenoxy)ethyl]acetamide

279. N-{2-[4-(6-chloro-7-{[1-(thiophen-2-ylmethyl)piperidin-4-yl]amino}-3H -imidazo[4,5-b]pyridin-2-yl)phenoxy]ethyl}acetamide

280. N-{2-[4-(6-chloro-7-{[1-(4-methoxybenzyl)piperidin-4-yl]amino}-3H -imidazo[4,5-b]pyridin-2-yl(phenoxy]ethyl}propanamide

281. N-{2-[4-(6-chloro-7-{[1-(4-methoxybenzyl)piperidin-4-yl]amino}-3H -imidazo[4,5-b]pyridin-2-yl)phenoxy]ethyl}cyclopentanecarboxamide

282. N-{2-[4-(6-chloro-7-{[1-(4-methoxybenzyl)piperidin-4-yl]amino}-3H-imidazo [4,5-b]pyridin-2-yl)phenoxy]ethyl}-2-methylpropanamide

283. N-{2-[4-(6-chloro-7-{[1-(4-methoxybenzyl)piperidin-4-yl]amino}-3H -imidazo[4,5-b]pyridin-2-yl)phenoxy]ethyl}pyridine-4-carboxamide

284. 2-{4-[6-Bromo-7-({1-[4-(1H-1,2,4-triazol-1-yl)benzyl]piperidin-4-yl}amino)-3H -imidazo[4,5-b]pyridin-2-yl]phenoxy}-N-methylacetamide

285. N-(2-{4-[6-chloro-7-({1-[4-(1H-1,2,4-triazol-1-yl)benzyl]piperidin-4-yl}amino)-3H-imidazo[4,5-b]pyridin-2-yl]phenoxy}ethyl)acetamide

286. N-(2-{4-[6-bromo-7-({1-[4-(1H-1,2,4-triazol-1-yl)benzyl]piperidin-4-yl}amino)-3H-imidazo[4,5-b]pyridin-2-yl]phenoxy}ethyl)acetamide

287. 2-{4-[6-chloro-7-({1-[(1,3,5-trimethyl-1H-pyrazol-4-yl)methyl]piperidin-4-yl}amino)-3H-imidazo[4,5-b]pyridin-2-yl]phenoxy}-N-methylacetamide

288. 2-{4-[6-bromo-7-({1-[(1,3,5-trimethyl-1H-pyrazol-4-yl)methyl]piperidin-4-yl}amino)-3H-imidazo[4,5-b]pyridin-2-yl]phenoxy}-N-methylacetamide

289. 2-[4-(6-chloro-7-{[1-(4-methoxybenzyl)piperidin-4-yl]amino}-3H-imidazo[4,5-b]pyridin-2-yl(phenoxy]-N-pyridin-3-ylacetamide

290. 2-[4-(6-chloro-7-{[1-(4-methoxybenzyl)piperidin-4-yl]amino}-3H-imidazo[4,5-b]pyridin-2-yl(phenoxy]-N-(1-methyl-1H-pyrazol-5-yl)acetamide

291. 2-(4-{6-chloro-7-[(1-methylpiperidin-4-yl)amino]-3H-imidazo[4,5-b]pyridin-2-yl}phenoxy)-N-pyridin-3-ylacetamide

292. 2-(4-{6-chloro-7-[(1-methylpiperidin-4-yl)amino]-3H-imidazo[4,5-b]pyridin-2-yl}phenoxy)-N-pyrazin-2-ylacetamide

293. N2-(4-{6-bromo-7-[(1-methylpiperidin-4-yl)amino]-3H-imidazo[4,5-b]pyridin-2-yl}phenyl)-N-methylglycinamide

294. N2-(4-(6-chloro-7-[(1-methylpiperidin-4-yl)amino]-3H-imidazo[4,5-b]pyridin-2-yl}phenyl)-N-methylglycinamide

295. N2-[4-(6-chloro-7-{[1-(1-methylethyl)piperidin-4-yl]amino}-3H-imidazo[4,5-b]pyridin-2-yl(phenyl]-N-methylglycinamide

296. N2-[4-(6-chloro-7-{[1-(4-methoxybenzyl)piperidin-4-yl]amino}-3H-imidazo[4,5-b]pyridin-2-yl)phenyl]-N-methylglycinamide

297. N2-(4-{6-chloro-7-[(1-ethylpiperidin-4-yl)amino]-3H-imidazo[4,5-b]pyridin-2-yl}phenyl)-N-methylglycinamide

298. 2-(4-{6-chloro-7-[(1-methylpiperidin-4-yl)amino]-3H-imidazo[4,5-b]pyridin-2-yl}phenoxy)-N-pyridin-2-ylacetamide

299. 2-(4-{6-chloro-7-[(1-methylpiperidin-4-yl)amino]-3H-imidazo[4,5-b]pyridin-2-yl}phenoxy)-N-isoxazol-3-ylacetamide

300. 2-(4-{6-chloro-7-[(1-methylpiperidin-4-yl)amino]-3H-imidazo[4,5-b]pyridin-2-yl}phenoxy)-N-(pyridin-4-ylmethyl)acetamide

301. N3-(4-{6-bromo-7-[(1-methylpiperidin-4-yl)amino]-3H-imidazo[4,5-b]pyridin-2-yl}phenyl)-N-methyl-b-alaninamide

302. N3-(4-{6-chloro-7-[(1-methylpiperidin-4-yl)amino]-3H-imidazo[4,5-b]pyridin-2-yl}phenyl)-N-methyl-b-alaninamide

303. N3-[4-(6-chloro-7-{[1-(1-methylethyl)piperidin-4-yl]amino}-3H-imidazo[4,5-b]pyridin-2-yl(phenyl]-N-methyl-b-alaninamide

304. N3-[4-(6-chloro-7-{[1-(4-methoxybenzyl)piperidin-4-yl]amino}-3H-imidazo[4,5-b]pyridin-2-yl)phenyl]-N-methyl-b-alaninamide

305. N3-(4-{6-chloro-7-[(1-ethylpiperidin-4-yl)amino]-3H-imidazo[4,5-b]pyridin-2-yl}phenyl)-N-methyl-b-alaninamide

306. 2-[4-(6-chloro-7-{[1-(4-methoxybenzyl)piperidin-4-yl]amino}-3H-imidazo[4,5-b]pyridin-2-yl(phenoxy]-N-(pyridin-4-ylmethyl)acetamide

307. 2-[4-(6-chloro-7-{[1-(4-methoxybenzyl)piperidin-4-yl]amino}-3H-imidazo[4,5-b]pyridin-2-yl(phenoxy]-N-pyrimidin-2-ylacetamide

308. 2-(4-{6-chloro-7-[(1-methylpiperidin-4-yl)amino]-3H-imidazo[4,5-b]pyridin-2-yl}phenoxy)-N-pyrimidin-2-ylacetamide

309. 2-[4-(6-chloro-7-{[1-(thiophen-2-ylmethyl)piperidin-4-yl]amino}-3H -imidazo[4,5-b]pyridin-2-yl)phenoxy]-N-pyrimidin-2-ylacetamide

310. 2-(4-{6-chloro-7-[(1-methylpiperidin-4-yl)amino]-3H-imidazo[4,5-b]pyridin-2-yl}phenoxy)-N-pyrazin-2-ylacetamide

311. 2-(4-{6-bromo-7-[(1-methylpiperidin-4-yl)amino]-3H-imidazo[4,5-b]pyridin-2-yl}phenoxy)-N-pyrazin-2-ylacetamide

312. 2-[4-(6-chloro-7-{[1-(1-methylethyl)piperidin-4-yl]amino}-3H-imidazo[4,5-b]pyridin-2-yl(phenoxy]-N-pyrazin-2-ylacetamide

313. 2-[4-(6-chloro-7-{[1-(thiophen-3-ylmethyl)piperidin-4-yl]amino}-3H -imidazo[4,5-b]pyridin-2-yl)phenoxy]-N-pyrazin-2-ylacetamide

314. 2-[4-(6-chloro-7-{[1-(2,3-dihydro-1-benzofuran-5-ylmethyl)piperidin-4-yl]amino}-3H-imidazo[4,5-b]pyridin-2-yl)phenoxy]-N-pyrazin-2-ylacetamide

315. 2-(4-{5-chloro-4-[(1-methylpiperidin-4-yl)amino]-1H-pyrrolo [2,3-b]pyridin-2-yl}phenoxy)-N-(5-methylisoxazol-3-yl)acetamide

316. N2-(4-{6-chloro-7-[(1-methylpiperidin-4-yl)amino]-3H-imidazo[4,5-b]pyridin-2-yl}phenyl)-N2-methyl-N-pyridin-3-ylglycinamide

317. 2-(4-{6-chloro-7-[(1-methylpiperidin-4-yl)amino]-3H-imidazo[4,5-b]pyridin-2-yl}phenoxy)-N-(5-chloropyridin-3-yl)acetamide

318. 2-[4-(6-chloro-7-{[1-(4-methoxybenzyl)piperidin-4-yl]amino}-3H-imidazo[4,5-b]pyridin-2-yl(phenoxy]-N-isoxazol-3-ylacetamide

319. 2-(4-{6-bromo-7-[(1-methylpiperidin-4-yl)amino]-3H-imidazo[4,5-b]pyridin-2-yl}phenoxy)-N-isoxazol-3-ylacetamide

320. 2-[4-(6-chloro-7-{[1-(1-methylethyl)piperidin-4-yl]amino}-3H-imidazo[4,5-b]pyridin-2-yl(phenoxy]-N-isoxazol-3-ylacetamide

321. 2-[4-(6-chloro-7-{[1-(thiophen-3-ylmethyl)piperidin-4-yl]amino}-3H -imidazo[4,5-b]pyridin-2-yl)phenoxy]-N-isoxazol-3-ylacetamide

322. 2-(4-{6-chloro-7-[(1-propylpiperidin-4-yl)amino]-3H-imidazo[4,5-b]pyridin-2-yl}phenoxy)-N-isoxazol-3-ylacetamide

323. 2-[4-(6-chloro-7-{[1-(4-methoxybenzyl)piperidin-4-yl]amino}-3H-imidazo[4,5-b]pyridin-2-yl(phenoxy]-N-pyrazin-2-ylacetamide

324. 2-(4-{6-chloro-7-[(1-methylpiperidin-4-yl)amino]-3H-imidazo[4,5-b]pyridin-2-yl}phenoxy)-N-(1-methyl-1H-pyrazol-5-yl)acetamide

325. 2-[4-(6-chloro-7-{[1-(4-methoxybenzyl)piperidin-4-yl]amino}-3H-imidazo[4,5-b]pyridin-2-yl(phenoxy]-N-1H-1,2,4-triazol-3-ylacetamide

326. 2-(4-{6-chloro-7-[(1-methylpiperidin-4-yl)amino]-3H-imidazo[4,5-b]pyridin-2-yl}phenoxy)-N-1H-1,2,4-triazol-3-ylacetamide

327. 2-(4-{6-chloro-7-[(1-methylpiperidin-4-yl)amino]-3H-imidazo[4,5-b]pyridin-2-yl}phenoxy)-N-1,3,4-thiadiazol-2-ylacetamide

328. 2-(4-{6-chloro-7-[(1-methylpiperidin-4-yl)amino]-3H-imidazo[4,5-b]pyridin-2-yl}phenoxy)-N-(3-methylisoxazol-5-yl)acetamide

329. 2-(4-{6-chloro-7-[(1-methylpiperidin-4-yl)amino]-3H-imidazo[4,5-b]pyridin-2-yl}phenoxy)-N-1,3-thiazol-2-ylacetamide

330. N-(5-tert-butylisoxazol-3-yl)-2-(4-{6-chloro-7-[(1-methylpiperidin-4-yl)amino]-3H-imidazo[4,5-b]pyridin-2-yl}phenoxy)acetamide

331. 2-(4-{6-chloro-7-[(1-niethylpiperidin-4-yl)amino]-3H-imidazo[4,5-b]pyridin-2-yl}phenoxy)-N-pyrimidin-5-ylacetamide

Preferred 2-phenyl-3H-imidazo[4,5-b]pyridine ROR1 inhibitors include numbers 2, 3, 11, 15, 19, 27, 34, 43, 56, 64, 71, 75, 82, 96, 99, 117, 132, 147, 159, 168, 184, 232, 259, 265, 272, 288, 294, 302, supra.

1. A method of treating glaucoma or pathogenic intraocular pressure, comprising administering to a person in need thereof an inhibitor of an ocular Wnt5a effector.

2. The method of claim 1 wherein the Wnt5a effector is selected from:
FZD2 (frizzled-2), FZD5 (frizzled-5) and ROR1 (Receptor Tyrosine Kinase Like Orphan Receptor 1); or
PLCB1 (phospholipase C, beta 1), PPP3R1 (Protein Phosphatase 3 Regulatory Subunit B, Alpha), NFATC3 (Nuclear Factor of Activated T Cells 3) and CAMK2D (Calcium/Calmodulin Dependent Protein Kinase II Delta).

2. The method of claim 1, wherein the inhibitor inhibits effector expression through a genetic manipulation selected from CRISPR gene editing and siRNA.

3. The method of claim 1, wherein the inhibitor inhibits the effector directly and is selected from an antibody, a small interfering peptide, and a small molecule inhibitor.

4. The method of claim 1, wherein the administering step comprises locally administering the inhibitor to an eye in need thereof.

5. The method of claim 1, wherein the administering step comprises delivery by eye drop or by intracameral administration or injection, subconjuctival administration or injection or intravitreal administration or injection.

6. The method of claim 1, wherein the administration is topical, and the inhibitor is administered in form of a topical ophthalmic gel, ointment, suspension or solution or contact lens.

7. The method of claim 1 wherein the inhibitor is a Wnt5a effector specific antibody.

8. The method of claim 1 wherein the inhibitor is a Wnt5a effector specific antibody that is cirmtuzumab.

9. The method of claim 1 wherein the inhibitor is a Wnt5a effector specific small molecule selected from compounds #1-#331 of Table KAN0439834 and related 2-phenyl-3H -imidazo[4,5-b]pyridine ROR1 inhibitors, supra.

10. The method of claim 1 wherein the inhibitor is a Wnt5a effector specific small molecule selected from compounds #2, 3, 11, 15, 19, 27, 34, 43, 56, 64, 71, 75, 82, 96, 99, 117, 132, 147, 159, 168, 184, 232, 259, 265, 272, 288, 294, 302 of Table KAN0439834 and related 2-phenyl-3H-imidazo[4,5-b]pyridine ROR1 inhibitors, supra.

11. The method of claim 1 wherein the inhibitor is a Wnt5a effector specific small molecule that is KAN0439834.

12. The method of claim 1 wherein the inhibitor is a Wnt5a effector specific siRNA and the effector is selected from ROR1, FZD2, FZD5, CAMK2D, PLCB1, PPP3R1 and NFATC3.

13. The method of claim 1 wherein the inhibitor is a Wnt5a effector specific siRNA and the effector is selected from ROR1, FZD2, FZD5, CAMK2D, PLCB1, PPP3R1 and NFATC3, and the siRNA comprises a sequences selected from SEQ ID 1-16 of Table, Human siRNA sequences, supra.

14. The method of claim 1, further comprising administering or coadministering locally at the eye a second, different inhibitor that is an inhibitor of an ocular Wnt5a effector.

15. The method of claim 7, wherein the administration is topical, and the inhibitor is administered in form of a topical ophthalmic gel, ointment, suspension or solution or contact lens.

16. The method of claim 8, wherein the administration is topical, and the inhibitor is administered in form of a topical ophthalmic gel, ointment, suspension or solution or contact lens.

17. The method of claim 9, wherein the administration is topical, and the inhibitor is administered in form of a topical ophthalmic gel, ointment, suspension or solution or contact lens.

18. The method of claim 11, wherein the administration is topical, and the inhibitor is administered in form of a topical ophthalmic gel, ointment, suspension or solution or contact lens.

19. The method of claim 12, wherein the administration is topical, and the inhibitor is administered in form of a topical ophthalmic gel, ointment, suspension or solution or contact lens.

20. The method of claim 13, wherein the administration is topical, and the inhibitor is administered in form of a topical ophthalmic gel, ointment, suspension or solution or contact lens; or
a composition that is an ophthalmic formulation of an inhibitor of an ocular Wnt5a effector, in unit dosage form, configured for a method for treating glaucoma or pathogenic intraocular pressure, the effector selected from:
FZD2 (frizzled-2), FZD5 (frizzled-5) and ROR1 (Receptor Tyrosine Kinase Like Orphan Receptor 1); or
PLCB1 (phospholipase C, beta 1), PPP3R1 (Protein Phosphatase 3 Regulatory Subunit B, Alpha), NFATC3 (Nuclear Factor of Activated T Cells 3) and CAMK2D (Calcium/Calmodulin Dependent Protein Kinase II Delta), and preferably:
the inhibitor inhibits effector expression through genetic manipulation, such as CRISPR gene editing or siRNA;
the inhibitor inhibits the effector directly and is selected from an antibody, a small interfering peptide, and a small molecule inhibitor;
in the form of a topical ophthalmic gel, ointment, suspension or solution;
the dosage form is an inhibitor-loaded contact lens, eye drop, depot or bollus;
packaged in an eye drop dispenser;
loaded in a syringe configured for intracameral administration or injection, subconjuctival administration or injection or intravitreal administration or injection;
further comprising excipients and features suitable for direct, topical delivery to the eye, selected from the group consisting of opthalmically suitable clarity, pH buffer, tonicity, viscosity, stability and sterility; and/or
the inhibitor is a ROR1 inhibitor, such as selected from cirmtuzumab and KAN0439834, or a FZD5 inhibitor, such as selected from anti-FZD5 antibodies IgG-2919 and IgG-2921, or a FZD2 inhibitor, such as selected from dFz7-21, a selective peptide, or FZD2 antibody or an siRNA such as disclosed herein.
